(19) United States
(12) Patent Application Publication
(10) Pub. No.: US 2025/0095774 A1
(43) Pub. Date: Mar. 20, 2025

(54) MACHINE LEARNING METHODS AND SYSTEMS FOR ACUTE RESPIRATORY DISTRESS SYNDROME PATIENT SUB-PHENOTYPING

(71) Applicant: GEN1E LIFESCIENCES INC., PALO ALTO, CA (US)

(72) Inventors: RITU LAL, PALO ALTO, CA (US); MAYA DAS, PALO ALTO, CA (US); RISHABH GOEL, PALO ALTO, CA (US); MRIDUL MEHTA, PALO ALTO, CA (US)

(21) Appl. No.: 18/890,241

(22) Filed: Sep. 19, 2024

Related U.S. Application Data
(60) Provisional application No. 63/539,246, filed on Sep. 19, 2023.

(51) Int. Cl. G16B 20/00; G16B 40/20; G16H 10/60; G16H 50/20

(52) U.S. Cl. CPC G16B 20/00; G16B 40/20; G16H 10/60; G16H 50/20

(57) ABSTRACT

Methods, systems, and apparatus, including computer programs encoded on computer storage media, for sub-phenotyping acute respiratory distress syndrome (ARDS) patients using machine learning are described. An example system may obtain training data comprising ARDS patient records, determine a number of ARDS patient sub-phenotypes based on the ARDS patient records using latent profile analysis, and then execute an iterative process for determining an optimal ARDS classifier for patient sub-phenotyping. The system may select the optimal ARDS classifier from the optimal modes of the plurality of machine learning classifiers by comparing the performance metrics and deploy the optimal ARDS classifier for ARDS patient sub-phenotyping and providing personalized treatment based on the optimal ARDS classifier.

This application claims the benefit under 35 U.S.C. § 119(e) of U.S. Provisional Application No. 63/539,246 filed on Sep. 19, 2023, which is incorporated by reference in its entirety.

### FIELD

The disclosure relates generally to machine learning for patient sub-phenotyping, and in particular, acute respiratory distress syndrome (ARDS) patient sub-phenotyping.

### BACKGROUND

ARDS (acute respiratory distress syndrome) is a severe lung condition characterized by sudden inflammation in the lungs, leading to fluid accumulation in the air sacs and severe breathing difficulties. Due to the diverse clinical presentation and varying responses to treatment, sub-phenotyping ARDS patients has become increasingly important in medical research and clinical practice. The primary reasons for sub-phenotyping ARDS patients include identifying diverse clinical features and severity of the condition, compiling a subset of suitable diagnostic information, and tailoring treatment strategies based on patient ARDS sub-phenotypes.

A challenge encountered when interpreting clinical trials has been the broad and heterogeneous criteria used to define ARDS. Existing solutions for interpreting the clinical data include clustering algorithms and machine learning-based procedures utilizing biomarkers such as plasma proteins and transcriptomic data, and ventilator-related data. However, one significant practical challenge with these current solutions is the limited accessibility of biomarker or ventilator-related data, especially in resource- and time-constrained situations. For example, obtaining biomarker data involves laboratory work, and the process can often take a significant amount of time. As another example, amid the COVID-19 pandemic, ventilator-related data was frequently unavailable due to the shortage of ventilators. As a result, rapid clinical assessment of ARDS sub-phenotypes remains limited, posing a significant challenge to optimizing treatment of ARDS patients.

### SUMMARY

According to the present invention methods for determining an optimal acute respiratory distress syndrome (ARDS) classifier for ARDS sub-phenotyping, comprise: obtaining training data comprising ARDS patient records, wherein each ARDS patient record comprises a plurality of clinical variables and a plurality of clinical outcomes; determining a number of ARDS sub-phenotypes based on the ARDS patient records using latent profile analysis; executing an iterative process to determine an optimal ARDS classifier for determining the number of ARDS sub-phenotypes, wherein the iterative process comprises: constructing a plurality of machine learning classifiers, wherein, the constructing comprises: configuring an output layer of each of a plurality of machine learning classifiers based on the number of ARDS sub-phenotypes; configuring an input layer of each of the plurality of machine learning classifiers to accept the plurality of clinical variables; and each of the machine learning classifiers independently comprises a plurality of hyperparameters and a plurality of model parameters; for each of the machine learning classifiers, training and validating the machine learning classifier using a grid search with multiple-fold cross-validation on the training data to determine an optimal mode of the machine learning classifier, wherein the plurality of clinical variables of the training data are used as training input, and the clinical outcomes of the training data are used as training labels; and determining performance metrics for the optimal mode of each machine learning classifier during the multiple-fold cross-validation; and identifying the optimal ARDS classifier based on the optimal modes for each machine learning classifier of the plurality of machine learning classifiers by comparing the performance metrics.

According to the present invention non-transitory computer-readable storage media are configured with instructions executable by one or more processors to cause the one or more processors to perform operations comprising: obtaining training data comprising ARDS patient records, wherein each ARDS patient record comprises a plurality of clinical variables and clinical outcomes of historical patients; determining a number of ARDS patient sub-phenotypes based on the ARDS patient records using latent profile analysis; executing an iterative process for determining an optimal ARDS classifier for ARDS patient sub-phenotyping, wherein the iterative process comprises: constructing a plurality of machine learning classifiers, wherein the constructing comprises: configuring an output layer of each of the plurality of machine learning classifiers based on the number of ARDS patient sub-phenotypes, configuring an input layer of each of the plurality of machine learning classifiers to accept point-of-care variables in the plurality of clinical variables, and each of the plurality of machine learning classifiers comprises a plurality of hyperparameters and a plurality of model parameters, for each of the plurality of machine learning classifiers, training and validating the machine learning classifier using a grid search with multiple-fold cross-validation on the training data to determine an optimal mode of the machine learning classifier, wherein the point-of-care variables in the training data are used as training input, and the clinical outcomes are used as training labels, and determining performance metrics for the optimal modes of the plurality of machine learning classifiers during the multiple-fold cross-validation; and identifying the optimal ARDS classifier from the optimal modes of the plurality of machine learning classifiers by comparing the performance metrics.

According to the present invention methods for ARDS sub-phenotyping, comprise: obtaining clinical data of an ARDS patient over a duration, wherein the clinical data comprises point-of-care variable values, and excludes variables associated with biomarkers and ventilator-based parameters; entering the obtained clinical data into a classifier trained for ARDS sub-phenotyping to generate an ARDS sub-phenotype classification result, wherein the ARDS sub-phenotype classification result comprises one of a plurality of ARDS sub-phenotypes; storing the obtained clinical data of the ARDS patient and the ARDS sub-phenotype classification result into an Electronic Medical Record (EMR) system; temporally tracking the obtained clinical and the ARDS sub-phenotype classification result; and generating an alert or notification when a new ARDS sub-phenotype classification result is different than a previous ARDS sub-phenotype classification result.

According to the present invention an optimal ARDS classifier is determined according to the method according to the present invention.

According to the present invention an ARDS sub-phenotype classification of an ARDS patient is determined using an optimal ARDS classifier according to the present invention.

According to the present invention methods of treating the ARDS patient are based on an ARDS sub-phenotype classification according to the present invention.

According to the present invention methods of generating an ARDS sub-phenotype trajectory map of an ARDS patient comprise: generating a first ARDS sub-phenotype classification result based on a first set of point-of-care clinical data for the ARDS patient; generating a second ARDS sub-phenotype classification result based on a second set of point-of-care clinical data for the ARDS patient; and generating an ARDS sub-phenotype classification trajectory map based on the first ARDS sub-phenotype classification result and the second ARDS sub-phenotype classification result.

According to the present invention methods of generating an ARDS sub-phenotype trajectory map of an ARDS patient comprising: providing a longitudinal dataset comprising values for each point-of-care clinical variable of a set of point-of-care clinical variables of an ARDS patient obtained at two or more times; generating an ARDS sub-phenotype classification result based on the longitudinal dataset; and generating an ARDS sub-phenotype classification trajectory map based on the ARDS sub-phenotype classification result corresponding to each of the two or more times.

According to the present invention an ARDS sub-phenotype trajectory map for an ARDS patient is generated using a method according to the present invention.

According to the present invention methods of treating ARDS in a patient comprise administering an ARDS treatment to the ARDS patient based on an ARDS sub-phenotype trajectory map according to the present invention.

According to the present invention systems comprise one or more processors and one or more non-transitory computer-readable memories coupled to the one or more processors and configured with instructions executable by the one or more processors comprising: an ARDS sub-phenotype classification module; an ARDS sub-phenotype trajectory mapping module; and an ARDS clinical decision support module.

According to the present invention a non-transitory computer-readable storage medium is configured with instructions executable by one or more processors to cause the one or more processors to perform operations comprising: generating a first ARDS sub-phenotype classification result based on a first set of point-of-care clinical data for the ARDS patient; generating a second ARDS sub-phenotype classification result based on a second set of point-of-care clinical data for the ARDS patient; and generating an ARDS sub-phenotype classification trajectory map based on the first ARDS sub-phenotype classification result and the second ARDS sub-phenotype classification result.

According to the present invention methods of determining an ARDS sub-phenotype of an ARDS patient comprise inputting a plurality of clinical variables into an optimal ARDS classifier according to the present invention; and determining an ARDS sub-phenotype classification result of the ARDS patient.

### BRIEF DESCRIPTION OF THE DRAWINGS

Those skilled in the art will understand that the drawings described herein are for illustration purposes only. The drawings are not intended to limit the scope of the present disclosure.

FIG. 1A and FIG. 1B shows definitions for clinical criteria used to assess ARDS patients.

FIG. 2 shows an example of a method for ARDS patient sub-phenotyping using a pruned machine learning model provided by the present disclosure.

FIGS. 3A and 3B show a prior art (FIG. 3A) machine learning ARDS patient sub-phenotyping method and an example of a tailored machine learning ARDS patient sub-phenotyping method provided by the present disclosure (FIG. 3B).

FIG. 4 shows an example of the physical deployment of a pruned machine learning model for ARDS patient sub-phenotyping provided by the present disclosure.

FIG. 5 shows a system diagram for a physical deployment of a pruned machine learning model for ARDS patient sub-phenotyping provided by the present disclosure.

FIGS. 6A-6C show examples of performance metrics for different machine learning models used to identify ARDS patient sub-phenotypes. an optimal ARDS classifier

FIG. 7 is a table showing examples of performance metrics for different machine learning models used to identify ARDS patient sub-phenotypes. an optimal ARDS classifier

FIG. 8 shows an example of sub-phenotype classification results generated by applying a pruned machine learning model provided by the present disclosure to data sets obtained for ARDS randomized control trials. clinical

FIG. 9 shows a flow diagram for an example of a method for ARDS patient sub-phenotyping using a pruned machine learning model provided by the present disclosure.

FIG. 10 shows an example of a computer system for implementing a pruned machine learning model provided by the present disclosure.

FIG. 11 shows standardized values of clinical variables for two ARDS patient sub-phenotypes from the MMIV-IV database determined using latent profile analysis.

FIG. 12 shows Kaplan Meier survival curves for the two ARDS patient sub-phenotypes identified using methods provided by the present disclosure.

FIG. 13 shows standardized values of clinical variables for two ARDS patient sub-phenotypes from randomized controlled clinical trials determined using latent profile analysis.

### DETAILED DESCRIPTION

Acute respiratory distress syndrome (ARDS) continues to cause high mortality and morbidity. The 2012 Berlin definition used ventilator-based variables to make the diagnosis of ARDS. The new 2023 Global Definition of ARDS³ has broadened the criteria allowing for a diagnosis based on non-biomarker and non-ventilator variables.

The heterogeneous presentation of ARDS into distinct clinical sub-phenotypes has been shown to impact patient outcomes. These sub-phenotypes often distinguish mild/moderate ARDS patients from severe ones but are distinct from non-ARDS specific severity scores such as Sequential Organ Failure Assessment (SOFA) and Acute Physiology and Chronic Health Assessment (APACHE). This has complicated the success of a one-size-fits-all therapeutic approach in clinical trials and has contributed to the absence of a United States Food and Drug Administration (FDA) approved treatment for ARDS.

Given the urgent need for effective ARDS therapies, it is crucial to adopt both prognostic and predictive enrichment strategies to tailor therapies towards specific sub-phenotypes of ARDS patients. Machine learning (ML) has been successfully used for patient phenotyping in transplantation and has other advantages, such as mapping patient trajectories by computing sub-phenotypes using longitudinal patient data. However, the current methods used to identify sub-phenotypes depend on complex, specialized biomarker data and include ventilator-based clinical variables, limiting real-time clinical applicability, particularly in resource-constrained settings.

A Machine Learning (ML) model is used to prospectively and accurately determine ARDS sub-phenotypes (per the 2023 Global Definition) using only readily accessible (non-ventilator, non-biomarker) clinical variables. Accurately classifying ARDS patients using easily collected data can assist physicians in real-time decision making. Moreover, the model can be easily applied to create ARDS sub-phenotype trajectories of patients over time, providing insights into treatment response and factors that may affect ARDS sub-phenotype transitions.

The present disclosure is directed to machine learning-driven systems suitable for real-time patient sub-phenotyping, specifically focusing on ARDS patient sub-phenotyping. The machine learning systems utilize non-biomarker and non-ventilator clinical variables, which facilitates continuous assessment and personalized treatment of ARDS patients.

Starting with the real-world MIMIC-IV dataset, ARDS patients using the 2023 Global Definition of ARDS collected at the time of admission to the ICU were identified. Next, mixture modeling was used to identify two distinct sub-phenotypes of ARDS patients who exhibit significantly different clinical outcomes. Finally, a model was developed that can assign a sub-phenotype to an ARDS patient prospectively and accurately using only eight readily available clinical variables.

Several studies have identified sub-phenotypes in the broad heterogenous ARDS population. These models were based on the Berlin 2012 definition and used large lists of clinical and ventilator variables, including some complex biomarkers that require specialized laboratory capabilities. Thus, the volume and difficulty in obtaining some of these inputs in real-time have precluded the applicability of these models in clinical settings. The 2023 Global Definition of ARDS expanded diagnosis to patients who are not on a ventilator, further reducing the applicability of these past models.

The present model prospectively establishes an ARDS sub-phenotype in ARDS patients using the 2023 Global Definition. The model can identify ARDS sub-phenotypes with high accuracy using only eight readily available clinical variables as input. The simplicity of the model means it can be used to classify ARDS patients in a clinical setting frequently, facilitating the ability to provide an insightful real-time trajectory of mortality risk and treatment response. A deeper analysis of such longitudinal ARDS patient sub-phenotype trajectories is also possible, which can help determine factors that affect transitions between ARDS sub-phenotypes and thereby guide treatment strategy. The model was trained and tested using baseline data from ARDS patients and was also found to be agnostic to the treatment or intervention being applied. Heterogeneity of Treatment Effect (HTE) interaction was studied in all five randomized control trial (RCT) datasets based on available treatment strategy. The identified ARDS sub-phenotypes showed no statistically significant interaction with any of the treatment strategies in all five RCT datasets (p-values ranging from 0.20 to 0.74). The RCT datasets only included longer-term mortality (90-180 days) data. Based on model design, it is possible that treatment effects using our model on shorter-term mortality may be more statistically significant compared to longer-term mortality as has also been found by others. Importantly, the simplicity of our model makes it possible to use longitudinal trajectories to study treatment effects over time. This has not been possible to do using existing models with their reliance on extensive clinical and biomarker data.

As with past studies, our model also identifies two distinct clinically meaningful ARDS sub-phenotypes. These sub-phenotypes exhibit remarkably different mortality rates with the more severe class exhibiting lower oxygenation, platelet count, hematocrit, and blood pressure alongside higher creatinine, heart rate, and white blood cell count. Although similar to the hypo- and hyper-inflammatory sub-phenotypes identified in past studies, the present sub-phenotypes are distinct because the disclosed model only uses simple clinical variables and no biomarker or ventilator variables. A key strength of the model is that it is trained on real-world data from MIMIC-IV. The unique combination of ML techniques and unbiased, heterogeneous training data has resulted in a robust model, which should generalize to the broad ARDS population as evidenced by the model's consistent performance on five (5) different RCT datasets.

Methods, systems, and apparatus for Acute Respiratory Distress Syndrome (ARDS) patient sub-phenotyping using a pruned machine learning model are disclosed. Patient sub-phenotyping refers to a concept used in healthcare and medical research to classify and categorize patients with a specific medical condition based, for example, on clinical, genetic, and/or environmental data. Sub-phenotype categorization can help to interpret the manifestations and predict the clinical trajectory of ARDS within a heterogeneous patient population.

The systems and methods provided by the present disclosure can be applied to various patient sub-phenotyping scenarios. However, the present disclosure is directed to systems and methods for sub-phenotyping ARDS patients. ARDS is a heterogeneous condition known for its varied manifestations, potentially culminating in severe respiratory failure with life-threatening consequences. Recent changes in the definition of ARDS, as outlined in the New Global Definition 2023, have shifted the diagnostic focus away from relying on ventilator-based parameters. Instead, the new criteria focus on non-ventilator parameters that are more readily accessible in the clinical setting. The Global Definition 2023 for ARDS presents an opportunity to identify and categorize ARDS even in resource-limited clinical settings.

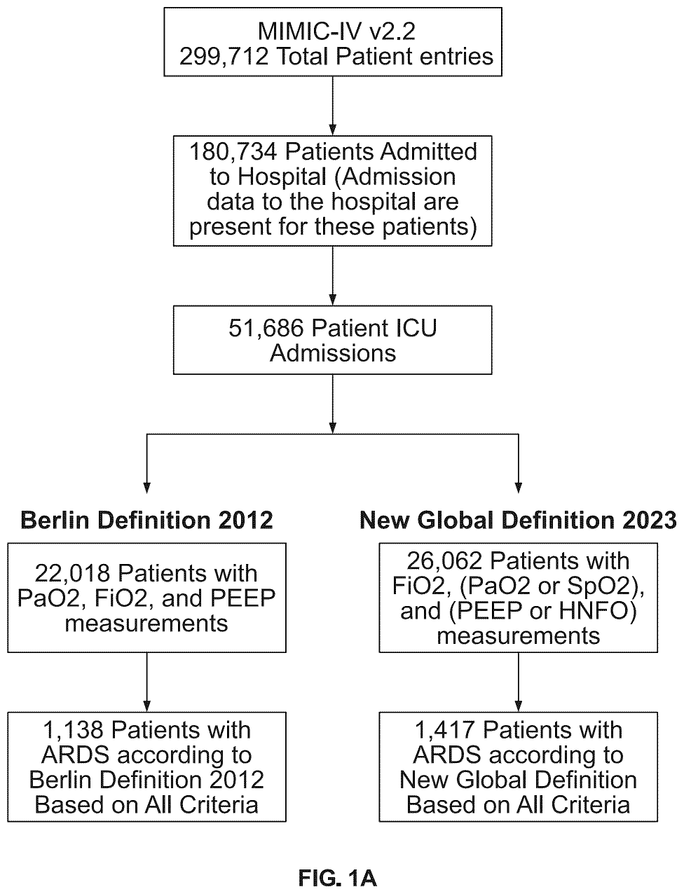

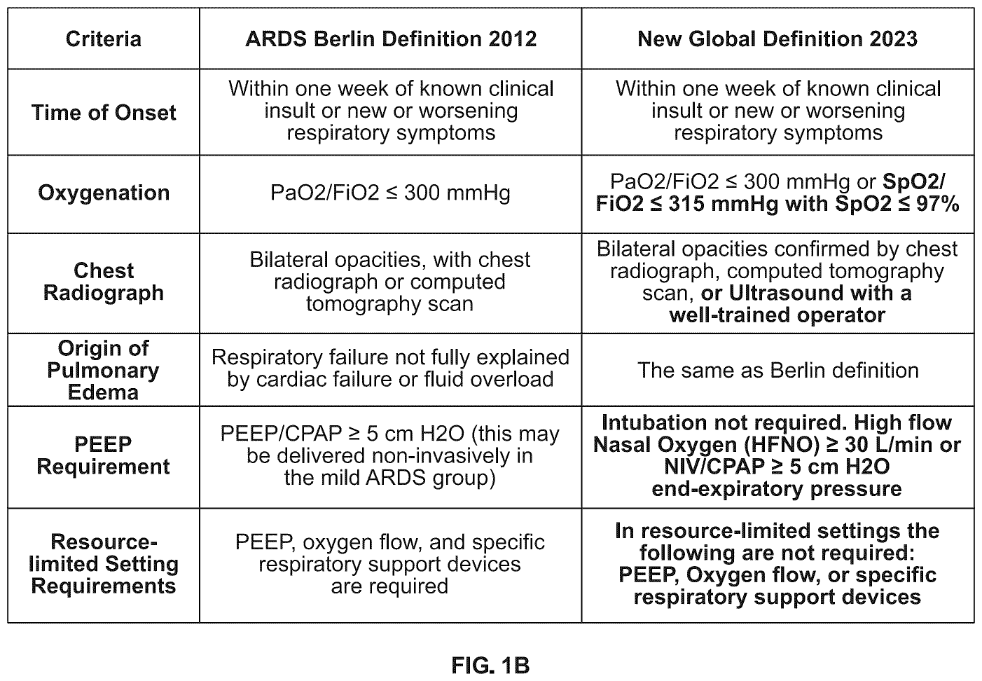

FIGS. 1A and 1B show ARDS definitions used to classify ARDS patients. As shown in FIG. 1A, MIMIC-IV (Medical Information Mart for Intensive Care IV) is a widely used, publicly available critical care database. The MIMIC-IV database contains health data for patients admitted to the Beth Israel Deaconess Medical Center in Boston, Massachusetts, USA, between 2008 and 2019. Among the 299,712 patients included in the MIMIC-IV database, hospital admission data were recorded for 180,734 patients. Within this group, 51,686 patients were admitted to the intensive care unit (ICU). Using the clinical data for the ICU patients, FIG. 1A depicts how the ARDS definition influences the diagnosis of the ARDS patients. Twenty-two thousand eighteen (22,0180 ICU patients in the MIMIC-IV database have values for the clinical variables identified by the previous ARDS definition (Berlin Definition 2012), which uses ventilator-based parameters such as the partial pressure of oxygen (PaO₂), the fraction of inspired oxygen (FiO₂), and the positive end-expiratory pressure (PEEP). Of these patients, one-thousand one-hundred and thirty-eight (1,138) patients meet the criteria for categorization as ARDS patients according to the Berlin Definition 2012 (FIG. 1B).

However, the newly published ARDS definition, New Global Definition 2023, uses different criteria for assessing ARDS. According to the criteria defined by the New Global Definition 2023 (FIG. 1B), 1,417 patients of the ICU patients in the MIMIC-IV database are categorized as ARDS patients.

FIG. 1B shows the ARDS classification criteria for the Berlin definition 2012 and for the New Global Definition 2023. For example, using the Berlin 2012 definition, ARDS patients in the MIMIC-IV dataset may be identified using the following procedure. First, structured query language (SQL) queries or scripts may be constructed to identify and obtain the patient records in the MIMIC-IV dataset that have measurements for PaO₂, FiO₂, and PEEP. For these patients, the oxygenation and PEEP variables for these patients may be compared with the corresponding criteria for the Berlin 2012 Definition. For example, ARDS patient candidates may be identified if the patient has (i) PaO₂/FiO₂≤300 mmHg and (ii) PEEP/CPAP≥5 cm H2O. For these ARDS patient candidates, free-text report notes associated with patient radiography imaging reports may be queried using SQL scripts from a second database such as MIMIC-IV-Note Database, to identify patients that also meet the Chest Radiograph criteria for the Berlin 2012 definition. Bilateral infiltrates may be confirmed using a natural language processing (NLP) model to analyze the free-text report notes. For example, the NLP model may search for synonymous keywords such as “bilateral infiltrates” or “bilateral consolidation.” As another example, the non-cardiac origin of pulmonary edema can be confirmed by searching keywords such as “non-cardiac,” “non-cardiogenic,” or “not cardiac.” Using this approach, 1,138 patients in the MIMIC-IV Database met all the criteria for the Berlin 2012 Definition of ARDS.

Consistent with the New Global Definition 2023 summarized in FIG. 1B, it is possible to diagnose ARDS patients even when PaO₂ measurements are not available and only SPO₂ measurements are provided. Similarly, ARDS patients can now be diagnosed in cases where PEEP measurements are absent and only HNFO (high flow nasal oxygen) measurements are recorded.

In the MIMIC-IV Database, FiO₂, (PaO₂ or SpO₂), and (PEEP or HNFO) measurements are recorded for 26,062 patients. For these patients, the oxygenation and PEEP criteria defined by the New Global Definition 2023 for ARDS are met when (i) PaO₂/FiO₂≤300 mmHg or SpO₂/FiO₂≤315 mmHg with an SPO₂≤97%; and (ii) PEEP/CPAP (continuous positive airway pressure)≥5 cm H₂O (cm of water pressure) or the HFNO≥30 L/min. For the patients meeting the oxygenation and PEEP criteria, SQL scripts may be constructed to query the free-text report notes associated with patient radiography imaging reports from the MIMIC-IV-Note Database to identify the patients meeting the Chest Radiograph criteria for the Berlin Definition 2012. A trained NLP model may be used to automatically parse the free-text report notes. For example, bilateral infiltrates can be confirmed by searching keywords such as “bilateral infiltrates” or “bilateral consolidation.” As another example, the non-cardiac origin of pulmonary edema can be confirmed by searching keywords such as “non-cardiac,” “non-cardiogenic,” or “not cardiac.” Using this method, 1,417 patients in the MIMIC-IV dataset met all criteria specified by the New Global 2023 Definition for ARDS.

The NLP model may be trained using preprocessed training data to learn the synonymous terms. For example, the training data may include a dataset that contains examples of terms consistent with the ARDS Definition together with synonymous terms. The dataset may then be labeled such that the meaning of each term is annotated with its corresponding synonyms. The text may be further preprocessed by removing stop words and performing other text cleaning operations to ensure consistency and quality of the training data. Then, the text may be converted into numerical representations using TF-IDF (Term Frequency-Inverse Document Frequency) or using word embeddings such as, for example, Word2Vec, GloVe, or FastText. These algorithms map words or phrases to vectors in high-dimensional spaces, capturing semantic relationships. Then a machine learning model may be selected for training. Models such as, for example, Conditional Random Fields (CRF), Bidirectional LSTM-CRF networks, or transformer-based models such as BERT (Bidirectional encoder Representations from Transformers) or GPT (Generative Pre-trained Transformer) may be selected. The training may be supervised and iterated for multiple rounds until the error rate is below a desired threshold.

The ARDS patient records identified in the MIMIC-IV Database and the MIMIC-IV-Note Database can serve as data for training and validating machine learning models to provide enhanced accuracy of ARDS patient sub-phenotyping.

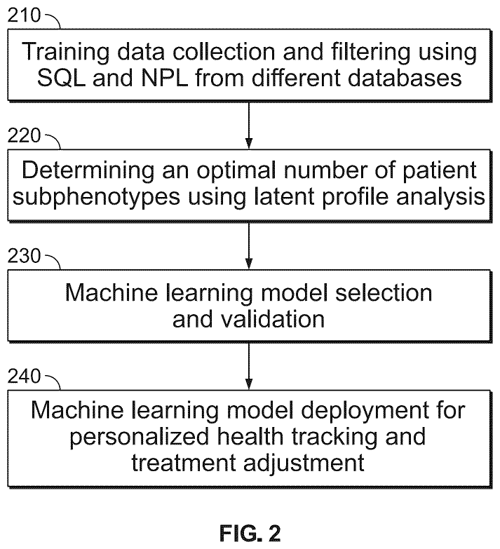

FIG. 2 shows a schematic of an example of a method for ARDS patient sub-phenotyping using a pruned machine learning model provided by the present disclosure. The method shown in FIG. 2 lists examples of steps that may be implemented in a centralized computer system, a distributed computer system, a cloud-based processing system, or in a software-as-a-service (SaaS) implementation, using containerization and orchestration, or edge computing. Depending on the implementation, the method may include fewer, more, or additional steps.

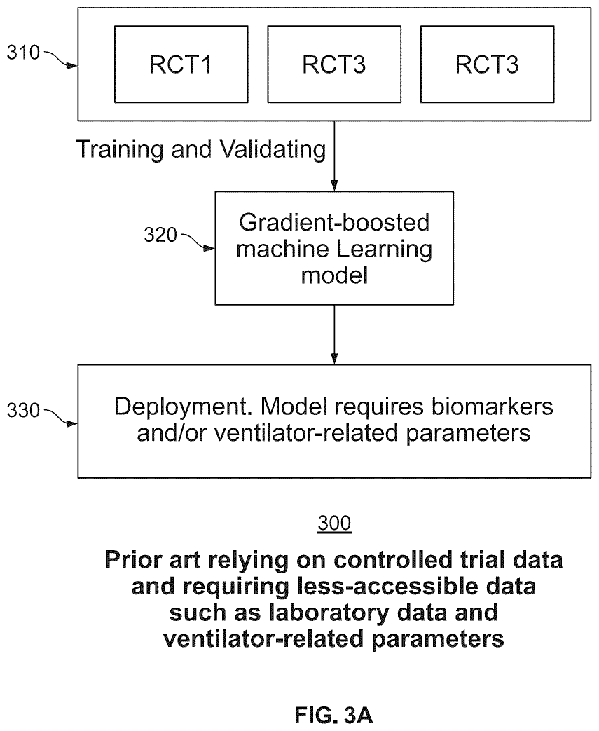

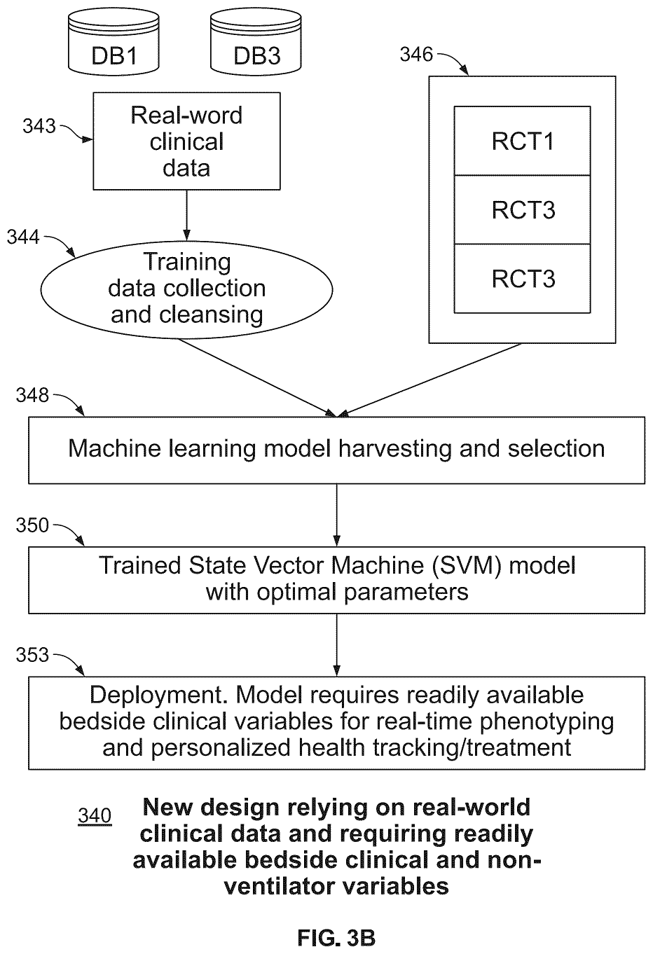

As shown in FIG. 2, step 210 includes training data collection and filtering using SQL and NLP from several ARDS patient databases. A detailed description of step 210 is provided in FIG. 3.

As shown in FIG. 2, after obtaining the ARDS patient records from step 210, step 220 includes determining an optimal number of ARDS patient sub-phenotypes based on the patient records using latent profile analysis (LPA). The optimal number of patient sub-phenotypes may determine the structure of the output layer of the ARDS patient sub-phenotyping machine learning model. LPA is a form of latent variable mixture modeling that enables the examination of subgroups of individuals within a larger heterogeneous population.

Prior ARDS classification methods have predominantly focused on categorizing ARDS patients consistent with the Berlin 2012 Definition and relied on biomarker data for clustering.

According to the methods provided by the present disclosure, the LPA can be applied to ARDS patients that meet the criteria of the updated New Global Definition 2023, i.e., the output of step 210. The LPA uses readily available bedside clinical and non-ventilator variables, ensuring that the results are relevant for intensive care unit (ICU) patient management and under resource-limited settings.

The LPA process may determine the optimal number of latent classes by assessing statistical and/or machine learning models containing/classifying from one to five classes. The quality of the model fit can be evaluated based on several metrics including, for example, the Bayesian information criterion (BIC), the integrated complete-data likelihood (ICL) criterion, the bootstrap likelihood ratio test (BLRT), class probability, or a combination of any of the foregoing. In certain embodiments, an “mclust” package in R programming language may be used to compute the BIC. ICL penalizes the BIC through an entropy term which measures class overlap. Higher ICL values are considered to have a better fit than lower ICL values. BLRT uses a bootstrap resampling method to approximate the p-value of the generalized likelihood ratio test comparing the K0-class mixture model with the K-1-class mixture model.

These metrics can be used to establish a balance between model complexity, fit, and class distinction clarity. This approach can ensure that the selected model accurately represents the underlying data structure without overfitting or underfitting. After selecting the optimal number of latent classes using the evaluation metrics, patients are assigned to a class that best corresponds to the clinical parameters of the patient based on the highest posterior probability of the model. Based on analysis of the MIMIC-IV databases the LPA shows that the optimal number of classes (sub-phenotypes) for ARDS patients is two. Class 1 has a higher 30-day mortality rate than Class 2, i.e., 38.73% vs 22.79% with p-value<0.01.

After determining the optimal number of classes, a machine learning model may undergo an iterative model harvesting process to determine a high-performance machine learning model for ARDS patient sub-phenotyping. For example, machine learning models or classifiers may be constructed, trained, validated, and evaluated. This process may use the training data obtained from step 210 (FIG. 2), and also other data that may have different characteristics than the training data to improve the generalizability of the machine learning models.

For example, the output layer of a machine learning classifier may be configured based on the optimal number of patient sub-phenotypes. The input layer of a machine learning classifier may be configured to accept point-of-care variables (physiological parameters excluding biomarkers and ventilator-related parameters) in the plurality of clinical variables. Because the machine learning models provided by the present disclosure exclude biomarkers and ventilator-related parameters, the input layer of the machine learning models are effectively pruned. In the following description, a pruned machine learning model is used to refer the new models relying on readily available bedside clinical and non-biomarker and non-ventilator variables.

Different machine learning models may require different construction methods. For example, Light Gradient Boosting Machine, Logistic Regression, Random Forest, Support Vector Machine, and Extreme Gradient Boosting may be constructed as classifiers and trained using the training data from step 210 (FIG. 2). Among these models, Light Gradient Boosting Machine, Logistic Regression, Random Forest are inherently binary classifiers. SVM constructs an optimal maximum-margin hyperplane in high-dimensional space for separation of classes. The hyperplane is positioned to maximize the distance to the nearest training points. This way of configuring SVM is effective at handling sparse high-dimensional patterns, even in the presence of outliers, by focusing on these support vectors rather than all points. The “output layer” of the SVM models may be configured to provide two class probabilities by setting the probability hyperparameter to True when initializing the SVM model. To provide two-class probabilities with XGBoost, the objective parameter may be set to “binary:logistic” for binary classification.

Each of these models includes two different sets of parameters: hyperparameters and model parameters. Hyperparameters are settings or configurations that are not learned from the data but are set before the training process begins. Hyperparameters control various aspects of the behavior of the model during training and optimization. Common hyperparameters include, for example, learning rate, batch size, the number of hidden layers in a neural network, the number of trees in a random forest, the depth of a decision tree, regularization strength (e.g., lambda in L1 or L2 regularization), and kernel choice in support vector machines. Model parameters are the internal variables or weights that a machine learning model learns from the training data. Model parameters define the relationships and patterns in the data that a model uses to make predictions.

To identify the optimal model with optimal hyperparameters and optimal model parameters, the model harvesting process in step 230 (FIG. 2) can include two layers of iterations: the outer layer of iteration iteratively tunes the hyperparameters of each model, and the inner layer of iteration iteratively learns the model parameters based on the given set of hyperparameters. For example, the two-layer iteration process may begin by establishing a matrix of potential hyperparameter values for the machine learning classifier. From this matrix, a specific combination of hyperparameters can be selected, and the machine learning classifier configured accordingly. The training data can then be partitioned into multiple subsets, denoted as N subsets (where N is greater than 1). The classifier undergoes training using N−1 of these subsets and performance validation is undertaken using the remaining subset, designated the validation subset. This training and validation cycle is repeated, with each cycle using a different subset of the training data as the validation set. The goal is to thoroughly assess the performance of the model across various data partitions. Subsequently, the process identifies the optimal model parameters for the machine learning classifier, which has been configured with the selected hyperparameter combination, based on the validation results. The procedure is then repeated using a different selected hyperparameter combination from the matrix and reconfiguring the machine learning classifier accordingly. Multiple rounds of training and validation are performed on this reconfigured classifier. The process is repeated until all combinations of hyperparameters are explored and evaluated. Ultimately, the process culminates in determining the most suitable mode of the machine learning classifier. This optimal mode includes the optimal hyperparameters and model parameters that provide the most favorable validation results.

During each round of inner iteration, the training may continue until the classification error rates are below a desired threshold. The classification error rates may be determined based on the labels of the training data (ground truth) and the model-generated classification results. For example, the ARDS patient records may be labeled during the process described in the LPA process. Each ARDS patient record may be labeled as either Class 1 (higher mortality rate) or Class 2 (lower mortality rate). Note that each individual ARDS patient record does not provide a mortality rate (it is either 0 or 1 for each individual patient). Therefore, the labeling process cannot be practically done by simply looking at the records for each individual ARDS patient. Instead, the labeling process involves using a statistical model or machine learning model to assign each ARDS patient to the class that best corresponds to the measured clinical parameters based on the highest posterior probability. This process requires iterative training and tuning to minimize overfitting or underfitting such as, for example, by applying regularization techniques such as L1 (Lasso) or L2 (Ridge) regularization to penalize the models with large coefficients, and hyperparameter tuning.

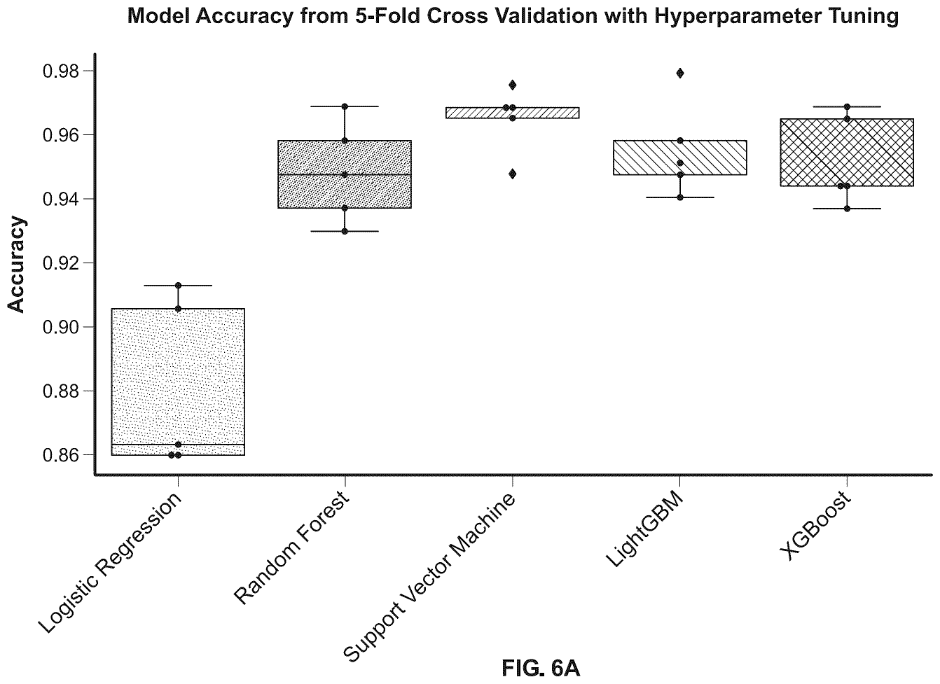

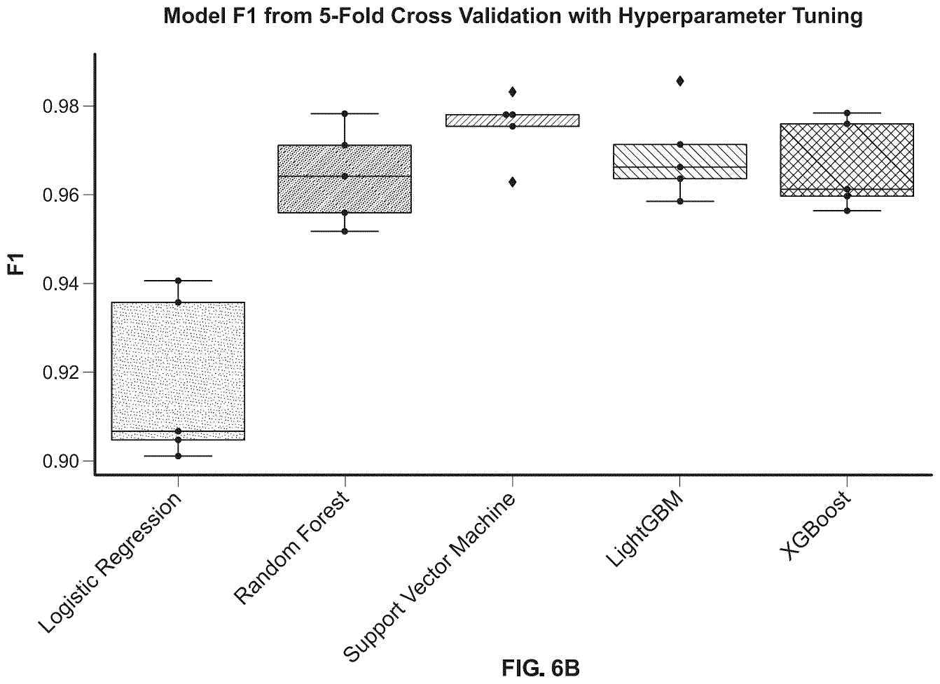

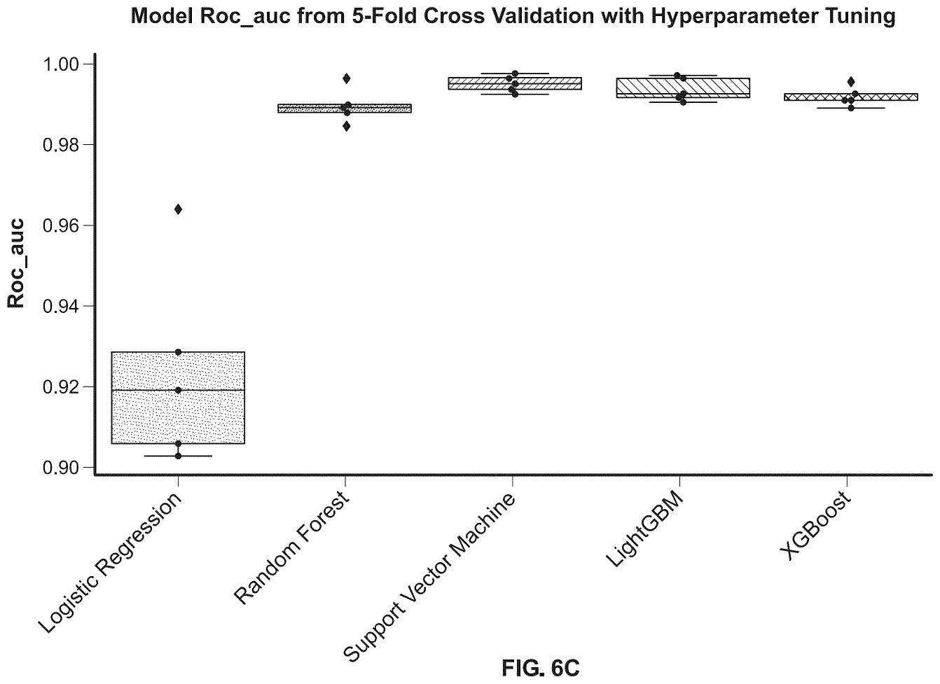

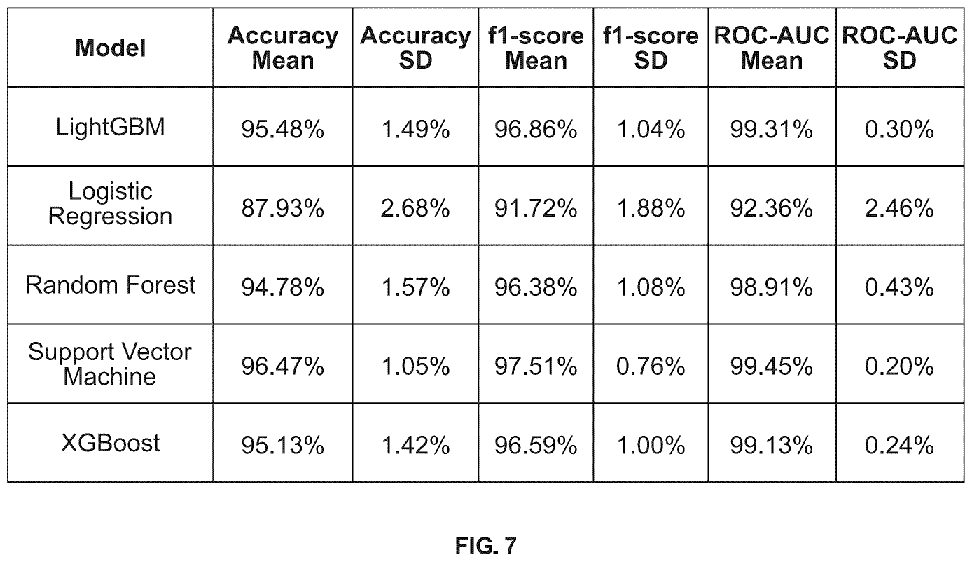

During the validation process in step 230 (FIG. 2), the performance metrics for each model are determined for each round of hyperparameter tuning and model parameter training. For example, the outer layer of iteration may utilize grid search and the inner layer of iteration may use 5-fold cross validation to determine the performance metrics for each model. The performance metrics can include, for example, accuracy, f1 score, ROC-AUC (Receiver Operating Characteristic-Area Under the Curve), or a combination of any of the foregoing. FIGS. 6A-6C and FIG. 7 show examples of performance metrics for different machine learning models. The model accuracy, f1 score, and ROC-AUC for the models in each of the 5 folds during the cross-validation are provided in the form of box-and-whisker graphs in FIGS. 6A-6C. The mean values of the performance metrics for each model across the 5 folds is provided in FIG. 7. As shown in FIG. 7, SVM has the highest mean across all metrics (accuracy=96.47%, f-1 Score=97.51%, and ROC-AUC=99.45%). SVM also shows the lowest SD (standard deviation) among the models in terms of accuracy, f1 score, and ROC-AUC. Therefore, using the training data collected from MIMIC-IV databases, SVM with the optimal hyperparameters and optimal model parameters may be selected as the optimal pruned-model for ARDS patient sub-phenotyping.

Referring to FIG. 2, after the pruned model is selected and using the optimal hyperparameters and the optimal model parameters, the pruned SVM model may be deployed for sub-phenotyping, diagnosing, and classifying ARDS patients.

FIGS. 3A and 3B show process flow diagrams for a prior art ARDS patient sub-phenotype classification method and for an example of a machine learning-based ARDS patient sub-phenotyping method using a pruned machine learning model provided by the present disclosure. The prior art process 300 includes a training-validation using a single data set, or data from similar sources with similar distributions. For example, the training and validation data 310 in the prior art 300 process represent multiple randomized controlled trial (RCT) cohorts. RCTs are carefully designed clinical trials with strict inclusion and exclusion criteria. Data from RCT cohorts are typically collected under controlled conditions and for a specific objective. RCTs provide high-quality, standardized, and well-documented information.

In the machine learning-based patient sub-phenotyping method provided by the present disclosure 340 clinical data sets from multiple MIMIC-IV databases are used as training data 343. The data compiled in the MIMIC-IV database is obtained during the course of routine clinical practice and ARDS patient care. The MIMIC-IV database reflects the diversity of patients, conditions, and treatments encountered in everyday healthcare settings. This data is less controlled and more varied compared to RCT data, which are often more homogeneous because they are designed to test specific interventions or treatments on a particular sub-group of ARDS patients who meet specific criteria. Using RCT data to train a machine learning model can limit the generalizability of the machine learning model. In addition, RCTs control for many variables, including treatment assignment and follow-up procedures. This controlled environment can minimize confounding factors, resulting in a tendency to attribute observed outcomes to specific clinical interventions. In contrast, data compiled in the MIMIC-IV database lack a similar level of control. ARDS patients receive treatments based on clinical judgment, and there may be multiple uncontrolled factors that influence outcomes. This can make it more challenging to isolate the effects of specific variables. Furthermore, although RCTs aim to minimize bias through randomization, the clinical trials may still introduce biases due to the selection of participants. On the other hands, data obtained during the course of clinical practice can reflect the complexities associated with patient care and therefore may have stronger external validity in the clinical setting compared to data obtained from RCTs. This means that models derived from data obtained in the clinical setting may be more applicable to broader patient populations and healthcare settings.

However, it is more challenging to convert clinical data into useable training data for training machine learning classifiers compared to using controlled or curated datasets. Clinical data is often collected from diverse sources, such as electronic health records (EHRs), medical images, sensor data, and patient-reported information. This heterogeneity can make it challenging to integrate and preprocess the data effectively. Clinical data may also suffer from missing values, inconsistencies, errors, and subjective assessments. Ensuring data quality and reliability is a critical and time-consuming task.

Referring to FIG. 3B, training data collection and data cleansing step 344 may start with constructing and executing a Structured Query Language (SQL) query against a designated first database such as MIMIC-IV, that contains patient monitoring records. This initial query is designed to identify ARDS patient candidates who exhibit a specific set of clinical parameters consistent with a diagnosis of ARDS. After the ARDS patient candidates are identified, the process proceeds to a second step. In this step, a second SQL query is constructed and executed against a separate second database such as MIMIC-IV-Note. The purpose of this second SQL query is to retrieve free-text report notes associated with radiography imaging reports for the identified patient candidates. These textual notes contain information relevant to an ARDS diagnosis. To further refine the identification of ARDS patients from the patient candidate population, a Natural Language Processing (NLP) model may be employed in the third step.

This NLP model is specifically trained to process and analyze the free-text report notes. The purpose of using the NLP model is to identify patients who meet a specific set of criteria outlined in the ARDS definition, thereby confirming ARDS diagnosis based on textual data. Note that the NLP training model involves supervised learning where the model is taught to recognize keywords and synonyms that are indicative of ARDS in the context of these textual notes.

Finally, after identifying ARDS patients with the second set of clinical parameters using the NLP model, the corresponding patient records may be extracted and labeled for training the pruned machine learning classifiers used for ARDS patient sub-phenotyping. The labeling process may involve using a statistical model or a machine learning model to assign each patient to the class that best matches the patient clinical parameters based on the highest posterior probability. Other preprocessing operations may be executed against the training data including performing log-transformation on non-normal variables in the patient records such that a distribution of variables in the patient records approximates a normal distribution, reducing data redundancy, or a combination thereof. Reducing data redundancy may include eliminating one variable of a pair of variables in the patient records with a correlation coefficient greater than a selected threshold. These steps effectively and significantly reduce the amount of data to be processed during the computing-intensive training process.

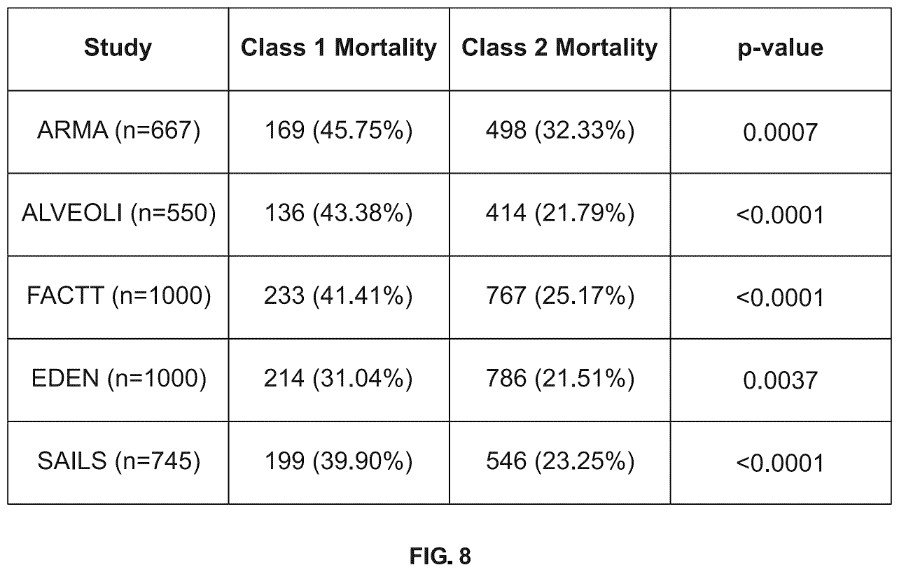

Another noticeable difference between the prior art process 300 and the machine learning-based patient sub-phenotyping method 340 provided by the present disclosure is that the method provided by the present disclosure 340 uses data from different sources for validation of the machine learning models. As shown in FIG. 3B, whereas the models in the patient sub-phenotyping methods 340 provided by the present disclosure are trained using real-world clinical data 343, the validation of the prior art models are from RTCs 346. The distributions of patient parameters may be similar within the RTCs 346, but they are different from distributions in the real-world clinical data 343. Using data with different parameter distributions for validation quantifies the generalizability of the models. FIG. 8 provides examples of test results generated using the pruned machine learning model provided by the present disclosure on various RTC datasets such as, for example, ARMA, ALVEOLI, FACTT, EDEN, and SAILS. The pruned machine learning model refers to a SVM model trained using the real-world clinical data. As shown, the pruned machine learning model can accurately classify the patients with low p-values, where Class 1 patients have a significantly higher mortality rate than Class 2 patients in all datasets, which is consistent with the pattern observed in the LPA analysis of the MIMIC-IV dataset.

Referring to FIG. 3A, the prior art method 300 uses a gradient-boosted machine learning model. The present method uses an SVM learning model. When dealing with high-dimensional data, SVM is advantageous. SVM is less prone to the “curse of dimensionality” compared to some ensemble methods and can still provide good generalization. Also, SVM outperforms the gradient-boosted machine learning model when the dataset is sparse and the imbalanced (class imbalance). The more direct reason for choosing SVM for use in the methods provided by the present disclosure is the results of the machine learning model harvesting step 348.

Furthermore, the machine learning model used in the prior art method 300, after deployment at step 330, requires biomarkers and/or ventilator-related clinical parameters, whereas the SVM model used in the methods provided by the present disclosure 340 relies on readily available bedside non-ventilator clinical variables for real-time sub-phenotyping and personalized health tracking and treatment.

In FIG. 3B, step 348 pertaining to machine learning model harvesting and selection and the step 350 pertaining to obtaining the optimal pruned SVM model with optimal hyperparameters and model parameters are described with respect to FIG. 2. The optimal pruned SVM model may generate two or more confidence scores corresponding to the number of ARDS patient-sub-phenotypes reflecting the values of the point-of-care non-ventilator variables. The deployment step 353 may include using the pruned SVM for ARDS diagnosis and for guiding treatment decisions of ARDS patients.

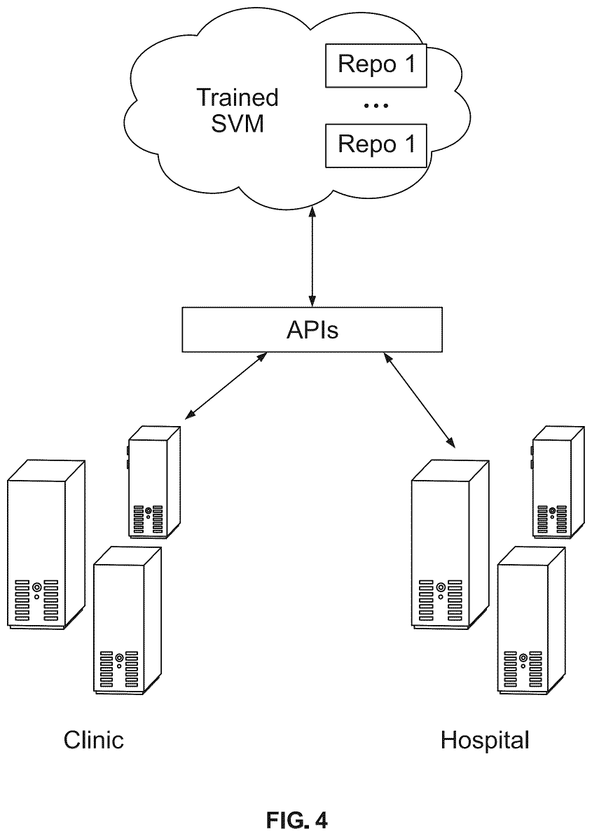

FIG. 4 shows an example of a physical deployment for a pruned machine learning model for ARDS patient sub-phenotyping provided by the present disclosure. The deployment shown in FIG. 4 can be adapted to suit specific needs or requirements.

As shown in FIG. 4, the pruned SVM model can be deployed in a cloud service. The cloud service may employ a plurality of application programming interfaces (APIs) to access the pruned SVM model. The plurality of APIs may include a set of input APIs for users to input patient data and a set of output APIs for outputting predicted patient ARDS sub-phenotype classifications. The cloud service may host a plurality of data repositories associated with different institutions such as clinics or hospitals. The data repositories may be understood as multi-tenants on the cloud. The data repositories may have different access controls to comply with data privacy regulations, data sharing agreements, and/or for technical considerations. In some scenarios, a user may want to improve the SVM model by providing more and new training data, and at the same time, preserve data privacy.

To achieve this goal, the system illustrated in FIG. 4 may implement federated or collaborative learning among institutions, in which the multiple institutions are allowed to collaboratively train the shared machine learning model, such as the pruned SVM model, while keeping proprietary data decentralized and localized. For example, instead of sending raw data to the central cloud server for model training, each institution can obtain a local copy of the model and train the model locally using proprietary data and then share only the model updates (gradients) with the central server. These updates may be aggregated to improve the global model, which can then be redistributed to all participants for further local training.

Instead of deploying the SVM model on a cloud service, the SVM model can be deployed within an institution. For instance, the SVM model may be integrated into an Electronic Medical Record (EMR) system that includes patient medical records, which displays a user interface (UI) element for entering clinical variables of the patient medical records into the SVM model and for receiving an ARDS sub-phenotype classification result.

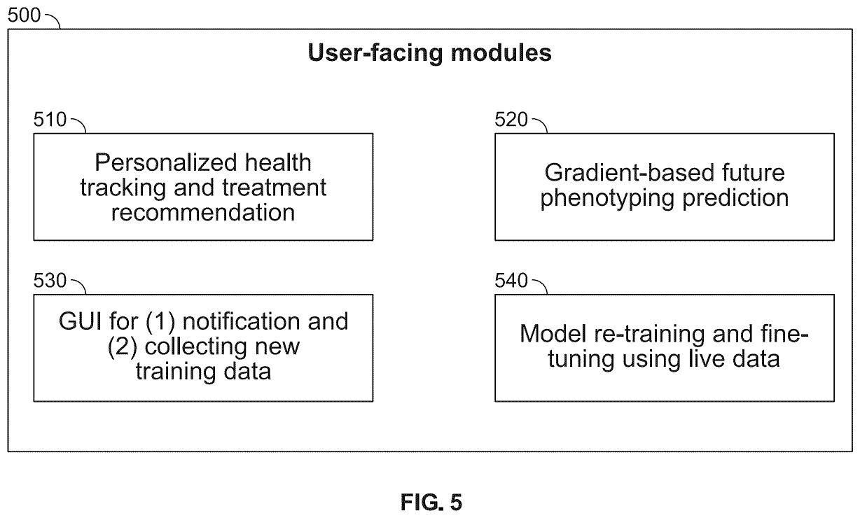

FIG. 5 shows an example of a system diagram for a deployment of a pruned machine learning model provided by the present disclosure. The system diagram includes multiple user-facing modules 500, such as a module 510 for personalized health tracking and treatment recommendation, a module 520 for gradient-based future sub-phenotyping prediction, a module 530 for GUI for (1) notification and (2) collecting new training data, and a module 540 for model re-training and fine-tuning using new clinical data.

Module 510 may be configured to temporally track the point-of-care non-biomarker and non-ventilator variables of a patient and a sequence of classification results that have been generated by the SVM model for the ARDS patient. An alert or notification may be generated when the variables of the patient trigger the SVM model to generate a new sub-phenotype classification result that is different from a previous sub-phenotype classification result. The change of sub-phenotype classification result may indicate recovery, progression, or response to medications, which may lead to a decision to cease or modify an ARDS treatment regimen.

Module 520 may be an enhanced version of module 510 and can be configured to predict a future ARDS sub-phenotype classification result for a patient. For example, module 520 may be configured to temporally track gradients of the point-of-care non-biomarker and non-ventilator clinical variables of the patient undergoing an ARDS treatment regimen and thereby facilitate prediction of future values of the clinical variables of the ARDS patient based on the tracked gradients. The future values of the clinical variables of the ARDS patient may be input into the SVM model for predicting a future ARDS sub-phenotype classification result for the patient. An alert or notification may be generated and displayed when the predicted future ARDS sub-phenotype classification result is different than a current ARDS sub-phenotype classification result.

Both (1) the classification results generated by the SVM model based on non-biomarker and non-ventilator ARDS patient clinical variables, and (2) explanatory information of the ARDS sub-phenotype classification result may be stored in a database. The module 530 may be a GUI for retrieving and displaying profile information of the ARDS patient and the alert or notifications showing the ARDS sub-phenotype of the patient. The GUI may include, for example, (a) a first UI element for providing access to the stored explanatory information, and (b) one or more additional UI elements for users to provide feedback on the ARDS sub-phenotype classification result. The feedback may indicate that the user agrees or disagrees with the ARDS sub-phenotype classification result generated by the SVM. In the case where the feedback indicates disagreement, the patient clinical variables and the feedback may be collected as a positive training sample, and the patient clinical variables and the ARDS sub-phenotype classification result may be collected as a negative training sample. The SVM model may later be fine-tuned or re-trained using the positive training sample and the negative training sample. Module 540 may carry out model-retraining and fine-tuning using real-time data collected from the user interactions.

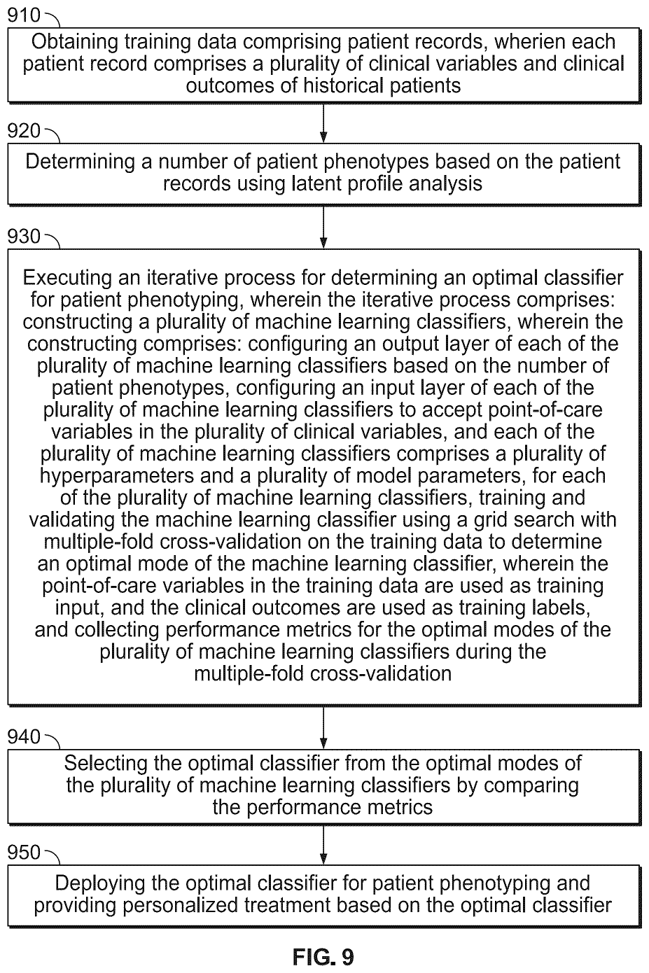

FIG. 9 shows an example of a method for ARDS patient sub-phenotyping using a pruned machine learning model provided by the present disclosure. The method 900 may be performed by a device, apparatus, or system. The method 900 may be performed by one or more modules/components of the environment or system shown in FIGS. 2-5. Depending on the implementation, the method 900 may include additional, fewer, or alternative steps performed in various orders or in parallel.

Step 910 includes obtaining training data comprising patient records, where each patient record comprises a plurality of clinical variables and clinical outcomes of historical ARDS patients.

Step 920 includes determining a number of ARDS patient sub-phenotypes based on the patient records using latent profile analysis.

Step 930 includes executing an iterative process for determining an optimal ARDS classifier for ARDS patient sub-phenotyping, wherein the iterative process comprises: constructing a plurality of machine learning classifiers, wherein the constructing comprises: configuring an output layer of each of the plurality of machine learning classifiers based on the number of patient sub-phenotypes, configuring an input layer of each of the plurality of machine learning classifiers to accept point-of-care variables in the plurality of clinical variables, and each of the plurality of machine learning classifiers comprises a plurality of hyperparameters and a plurality of model parameters, for each of the plurality of machine learning classifiers, training and validating the plurality of machine learning classifiers using a grid search with multiple-fold cross-validation on the training data to determine an optimal mode of the plurality of machine learning classifiers, wherein the point-of-care variables in the training data are used as training input, and the clinical outcomes are used as training labels, and collecting performance metrics for the optimal modes of the plurality of machine learning classifiers during the training and validating using multiple-fold cross-validation.

Step 940 includes selecting the optimal ARDS classifier from the optimal modes of the plurality of machine learning classifiers by comparing the performance metrics. The optimal ARDS classifier can be used to generate an ARDS sub-phenotype classification for an ARDS patient.

Step 950 includes deploying the optimal ARDS classifier for ARDS patient sub-phenotyping and generating a recommended treatment based on the ARDS sub-phenotype.

Aspects of the present disclosure include an optimal ARDS classifier such as an optimal ARDS classifier generated by a method according to the present disclosure. The optimal ARDS classifier can be used to determine an ARDS sub-phenotype of an ARDS patient. The ARDS sub-phenotype can be classified as a Class 1 ARDS sub-phenotype corresponding to a higher mortality risk or as a Class 2 ARDS sub-phenotype corresponding to a lower mortality risk. An ARDS sub-phenotype can be determined by inputting values for a plurality of clinical variables such as point-of care variables into the optimal ARDS classifier.

The point-of-care clinical variables can include heart rate, temperature, mean arterial blood pressure, SPO₂, blood urea nitrogen, albumin, bilirubin, serum creatinine, whole blood creatinine, serum glucose, whole blood glucose, serum hematocrit, whole blood hematocrit, PCO₂ platelets, sodium, arterial TCO₂, venous TCO₂, white blood cell count, or a combination of any of the foregoing. The point-of-care clinical variables can comprise heart rate, SPO₂, serum creatinine, serum glucose, serum hematocrit, platelets, sodium, and white blood cell count. The point-of-care clinical variables can consist of heart rate, SPO₂, serum creatinine, serum glucose, serum hematocrit, platelets, sodium, and white blood cell count.

A Class 2 low mortality risk ARDS sub-phenotype can be associated with, for example, a reduced risk of hospital mortality, reduced risk of ICU mortality, reduced risk of 28-day mortality, reduced risk of 90-day mortality, reduced risk of 180-day mortality, and reduced risk of 6-month mortality relative to a Class 1 high mortality risk sub-phenotype. A Class 2 low mortality risk ARDS sub-phenotype can be associated with a positive patient outcome such as, for example, at least one of a shorter hospital length of stay, a shorter ICU length of stay, and more ventilator-free days relative to negative patient outcome.

Methods provided by the present disclosure include treating ARDS in patient based on the determined ARDS sub-phenotype classification. For an ARDS patient identified by a Class 1 ARDS sub-phenotype classification a therapy recommendation can comprise, for example, one or more of neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, or full or trophic enteral feeding. For an ARDS patient identified by a Class 2 ARDS sub-phenotype classification a therapy recommendation can comprise, for example, one or more of NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, or full enteral feeding.

Other methods of ARDS treatment include, for example, airway pressure release ventilation, continuous positive airway pressure, driving pressure, extracorporeal membrane oxygenation, extracorporeal carbon dioxide removal, fluid management, glucocorticoid administration, high-flow nasal oxygen, high-frequency oscillatory ventilation, inhaled pulmonary vasodilators, low tidal volume ventilation, lung-protective ventilation, mechanical power adjustment, neuromuscular blocking agents, plateau pressure limitation, positive end expiratory pressure (peep) titration, prone positioning, recruitment maneuvers, systemic corticosteroids, tidal volume limitation, veno-venous extracorporeal membrane oxygenation, and ventilatory support. As appropriate a subset of these and other interventions can be undertaken to treat an ARDS patient identified by a Class 1 ARDS phenotype or a Class 2 ARDS phenotype.

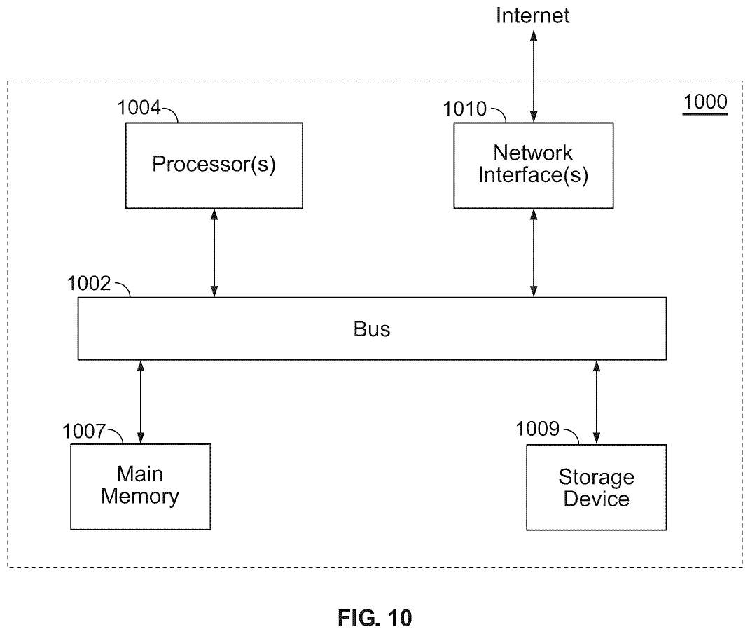

FIG. 10 illustrates an example of a computer system in which methods provided by the present disclosure may be implemented. The computing device may be used to implement one or more aspects of the systems and the methods shown in FIGS. 1-9. The computing device 1000 may comprise a bus 1002 or other communication mechanisms for communicating information and one or more hardware processors 1004 coupled with bus 1002 for processing information. Hardware processor(s) 1004 may be, for example, one or more general-purpose microprocessors.

The computing device 1000 can include a main memory 1007, such as random-access memory (RAM), cache and/or other dynamic storage devices, coupled to bus 1002 for storing information and instructions to be executed by processor(s) 1004. Main memory 1007 can be used for storing temporary variables or other intermediate information during execution of instructions to be executed by processor(s) 1004. Such instructions, when stored in storage media accessible to processor(s) 1004, can render computing device 1000 into a special-purpose machine that is customized to perform the operations specified in the instructions. Main memory 1007 can include non-volatile media and/or volatile media. Non-volatile media can include, for example, optical or magnetic disks. Volatile media can include dynamic memory. Common forms of media include, for example, a floppy disk, a flexible disk, a hard disk, a solid-state drive, a magnetic data storage medium such as magnetic tape, an optical data storage medium such as a CD-ROM, any physical medium with patterns of holes, a RAM, a DRAM, a PROM, and EPROM, a FLASH-EPROM, NVRAM, any other memory chip or cartridge, or networked versions of any of the foregoing.

The computing device 1000 may implement methods provided by the present disclosure using customized hard-wired logic, one or more ASICs or FPGAs, firmware and/or program logic which in combination with the computing device may cause or program computing device 1000 to be a special-purpose machine. Methods provided by the present disclosure can be performed by computing device 1000 in response to processor(s) 1004 executing one or more sequences of one or more instructions contained in main memory 1007. Such instructions can be read into main memory 1007 from another storage medium, such as storage device 1009. Execution of the sequences of instructions contained in main memory 1007 may cause processor(s) 1004 to perform the process steps described herein. For example, the processes/methods disclosed herein can be implemented by computer program instructions stored in main memory 1007. When these instructions are executed by processor(s) 1004, they can perform the steps as shown in corresponding figures and described above. In alternative embodiments, hard-wired circuitry can be used in place of or in combination with software instructions.

The computing device 1000 can include a communication interface 1010 coupled to bus 1002. Communication interface 1010 can provide a two-way data communication coupling to one or more network links that are connected to one or more networks. As another example, communication interface 1010 can be a local area network (LAN) card to provide a data communication connection to a compatible LAN (or WAN component to communicated with a WAN). Wireless links can also be implemented.

The performance of certain of the operations may be distributed among multiple processors, not only residing within a single machine, but deployed across several machines. For example, the processors or processor-implemented engines can be in a single geographic location such as within a home environment, an office environment, and/or a server farm. The processors or processor-implemented engines can be distributed across several geographic locations.

Each of the processes, methods, and algorithms provided by the present disclosure can be embodied in, and fully or partially automated by, code modules executed by one or more computer systems or computer processors comprising computer hardware. The processes and algorithms can be implemented partially or wholly in application-specific circuitry.

When the methods provided by the present disclosure are implemented in the form of software functional units and sold or used as independent products, the software functional units can be stored in a processor executable non-volatile computer-readable storage medium. Methods provided by the present disclosure, in whole or in part, or aspects that contribute to current technologies may be embodied in the form of a software product. The software product can be stored in a storage medium comprising instructions to cause a computing device, which may be a personal computer, a server, or a network device, to execute all or some of the steps of the methods provided by the present disclosure. A storage medium can comprise, for example, a flash drive, a portable hard drive, ROM, RAM, a magnetic disk, an optical disc, another medium operable to store program code, or a combination of any of the foregoing.

Aspects of the present disclosure include a system comprising a processor and a non-transitory computer-readable storage medium storing instructions executable by the processor to cause the system to perform operations corresponding to steps in any method of the embodiments disclosed above. Other aspects further provide a non-transitory computer-readable storage medium configured with instructions executable by one or more processors to cause the one or more processors to perform operations corresponding to steps in any method of the embodiments disclosed above.

Methods provided by the present disclosure may be implemented through a cloud platform, a server or a server group (hereinafter collectively the “service system”) that interacts with a client. The client can be a terminal device, or a client registered by a user at a platform, wherein the terminal device may be a mobile terminal, a personal computer (PC), and any device that may be installed with a platform application program.

The various features and processes provided by the present disclosure can be used independently of one another or can be combined in various ways. All possible combinations and sub-combinations are intended to fall within the scope of this disclosure. In addition, certain method or process blocks can be omitted in some implementations. The methods and processes provided by the present disclosure are also not limited to any particular sequence and the blocks or states relating thereto can be performed in other sequences that are appropriate. For example, described blocks or states can be performed in an order other than that specifically disclosed, or multiple blocks or states can be combined in a single block or state. The example blocks or states can be performed in serial, in parallel, or in some other manner. Blocks or states can be added to or removed from the disclosed example embodiments. The systems and methods provided by the present disclosure can be configured differently than described. For example, elements can be added to, removed from, or rearranged with respect to the disclosed examples.

Various operations of methods provided by the present disclosure can be performed, at least partially, by an algorithm. An algorithm can comprise program codes or instructions stored in a memory, such as a non-transitory computer-readable storage medium described above. An algorithm can comprise a machine learning algorithm. A machine learning algorithm cannot explicitly program computers to perform a function but can learn from training data to make a prediction model that performs the function.

Various operations of methods provided by the present disclosure can be performed, at least partially, by one or more processors that are temporarily configured, such as by software, or permanently configured to perform the relevant operations. Whether temporarily or permanently configured, such processors may constitute processor-implemented engines that operate to perform one or more operations or functions provided by the present disclosure.

Methods provided by the present disclosure can be at least partially processor-implemented, with a particular processor or processors being an example of hardware. For example, at least some of the operations of a method can be performed by one or more processors or processor-implemented engines. Moreover, the one or more processors can also operate to support performance of the relevant operations in a “cloud computing” environment or as a “software as a service” (SaaS). For example, at least some of the operations can be performed by a group of computers as an example of machines including processors, with these operations being accessible via a network (e.g., the Internet) and via one or more appropriate interfaces such as an Application Program Interface (API).

The performance of certain of the operations can be distributed among the processors, not only residing within a single machine, but deployed across several machines. The processors or processor-implemented engines can be located, for example, in a single geographic location such as within a home environment, an office environment, or a server farm. As another example, the processors or processor-implemented engines can be distributed across several geographic locations.

Throughout this specification, plural instances can implement components, operations, or structures described as a single instance. Although individual operations of one or more methods are illustrated and described as separate operations, one or more of the individual operations can be performed concurrently, and nothing requires that the operations be performed in the order illustrated. Structures and functionality presented as separate components in example configurations can be implemented as a combined structure or component. Similarly, structures and functionality presented as a single component can be implemented as separate components. These and other variations, modifications, additions, and improvements fall within the scope of the subject matter herein.

As used herein, “or” is inclusive and not exclusive, unless expressly indicated otherwise or indicated otherwise by context. Therefore, herein, “A, B, or C” means “A, B, A and B, A and C, B and C, or A, B, and C,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context. Moreover, plural instances may be provided for resources, operations, or structures described herein as a single instance. Additionally, boundaries between various resources, operations, engines, and data stores are somewhat arbitrary, and certain operations are illustrated in a context of specific illustrative configurations. Other allocations of functionality are envisioned and can fall within a scope of various embodiments of the present disclosure. In general, structures and functionality presented as separate resources in the example configurations can be implemented as a combined structure or resource. Similarly, structures and functionality presented as a single resource can be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The term “includes” or “comprises” is used to indicate the existence of the subsequently declared features but does not exclude the addition of other features. Conditional language, such as, among others, “can,” “could,” “might,” or “may,” unless specifically stated otherwise, or otherwise understood within the context as used, is intended to convey that certain embodiments include, while other embodiments do not include, certain features, elements and/or steps. Thus, such conditional language is not intended to imply that features, elements and/or steps are in any way required for one or more embodiments or that one or more embodiments necessarily include logic for deciding, with or without user input or prompting, whether these features, elements and/or steps are included or are to be performed in any particular embodiment.

### ASPECTS

The invention is further defined by one or more of the following aspects.

Aspect 1. A method for determining an optimal acute respiratory distress syndrome (ARDS) classifier for ARDS sub-phenotyping, comprising: obtaining training data comprising ARDS patient records, wherein each ARDS patient record comprises a plurality of clinical variables and a plurality of clinical outcomes; determining a number of ARDS sub-phenotypes based on the ARDS patient records using latent profile analysis; executing an iterative process to determine an optimal ARDS classifier for determining the number of ARDS sub-phenotypes, wherein the iterative process comprises: constructing a plurality of machine learning classifiers, wherein, the constructing comprises: configuring an output layer of each of a plurality of machine learning classifiers based on the number of ARDS sub-phenotypes; configuring an input layer of each of the plurality of machine learning classifiers to accept the plurality of clinical variables; and each of the machine learning classifiers independently comprises a plurality of hyperparameters and a plurality of model parameters; for each of the machine learning classifiers, training and validating the machine learning classifier using a grid search with multiple-fold cross-validation on the training data to determine an optimal mode of the machine learning classifier, wherein the plurality of clinical variables of the training data are used as training input, and the clinical outcomes of the training data are used as training labels; and determining performance metrics for the optimal mode of each machine learning classifier during the multiple-fold cross-validation; and identifying the optimal ARDS classifier based on the optimal modes for each machine learning classifier of the plurality of machine learning classifiers by comparing the performance metrics.

Aspect 2. The method of aspect 1, wherein the number of ARDS sub-phenotypes is two.

Aspect 3. The method of any one of aspects 1 to 2, wherein the plurality of clinical variables does not include clinical variables associated with biomarkers and clinical variables associated with ventilator-based parameters.

Aspect 4. The method of any one of aspects 1 to 3, wherein the plurality of clinical variables comprises a plurality of point-of-care clinical variables.

Aspect 5. The method of aspect 4, wherein the point-of-care clinical variables comprise a plurality of physiological parameters.

Aspect 6. The method of any one of aspects 4 to 5, wherein the point-of-care clinical variables comprise platelet concentration, SpO₂, serum creatinine concentration, serum hematocrit value, white blood cell count, sodium concentration, heart rate, serum glucose concentration, or a combination of any of the foregoing.

Aspect 7. The method of any one of aspects 4 to 5, wherein the point-of-care clinical variables comprise platelet concentration, SpO₂, serum creatinine concentration, serum hematocrit value, white blood cell count, sodium concentration, heart rate, and serum glucose concentration.

Aspect 8. The method of any one of aspects 4 to 5, wherein the point-of-care clinical variables consist of platelet concentration, SpO₂, serum creatinine concentration, serum hematocrit value, white blood cell count, sodium concentration, heart rate, and serum glucose concentration.

Aspect 9. The method of any one of aspects 4 to 5, wherein the plurality of clinical outcomes comprises mortality data.

Aspect 10. The method of any one of aspects 1 to 9, wherein the optimal mode of the machine learning classifier comprises optimal hyperparameters and optimal model parameters of the machine learning classifier, wherein, the optimal hyperparameters are used to configure the training of the machine learning classifier; and the optimal model parameters are adjusted during the training of the optimal model parameters.

Aspect 11. The method of any one of aspects 1 to 10, wherein the performance metrics comprise accuracy, F1 score, ROC-AUC (Receiver Operating Characteristic-Area Under the Curve), or a combination of any of the foregoing.

Aspect 12. The method of any one of aspects 1 to 11, wherein identifying the optimal ARDS classifier comprises selecting the machine learning classifier of the plurality of machine learning classifiers having the highest ROC-AUC compared to the other of the plurality of machine learning classifiers.

Aspect 13. The method of any one of aspects 1 to 12, wherein the plurality of machine learning classifiers comprises a Light Gradient Boosting Machine algorithm, a Logistic Regression algorithm, a Random Forest algorithm, a Support Vector Machine algorithm, an Extreme Gradient Boosting algorithm, or a combination of any of the foregoing.

Aspect 14. The method of any one of aspects 1 to 13, wherein the optimal ARDS classifier is a Support Vector Machine algorithm.

Aspect 15. The method of any one of aspects 1 to 14, before determining the number of ARDS sub-phenotypes, performing data cleaning and data preprocessing on the ARDS patient records to obtain the training data.

Aspect 16. The method of aspect 15, wherein the data cleaning comprises subjecting the data to multiple imputation using a chained equation method.

Aspect 17. The method of any one of aspects 15 to 16, wherein the data preprocessing comprises: performing log-transformation on the values for the plurality of clinical variables of the ARDS patient records such that a distribution of the values for each clinical variable approximates a normal distribution; reducing data redundancy; or a combination thereof.

Aspect 18. The method of aspect 17, wherein the normal distribution of values for each point-of-care clinical variable has a mean of zero and a standard deviation of one.

Aspect 19. The method of any one of aspects 17 to 18, wherein reducing data redundancy comprises, for each pair of variables in the ARDS patient record with a correlation coefficient greater than a threshold, eliminating one variable of the pair of variables in the ARDS patient record.

Aspect 20. The method of any one of aspects 1 to 19, wherein the training and validating the machine learning classifier using a grid search with multiple-fold cross-validation on the training data comprises: creating a matrix of candidate hyperparameter values for the machine learning classifier; selecting a first combination of hyperparameters values from the matrix of candidate hyperparameter values and configuring the machine learning classifier using the selected first combination of hyperparameter values; dividing the training data into N subsets, where N is an integer greater than 1; training the machine learning classifier using N−1 subsets of the N subsets and validating the trained machine learning classifier using a remaining subset of the N subsets as a validation subset; repeating the training and validation of the machine learning classifier for multiple rounds, wherein each round employs a different subset of the N subsets as the validation subset; determining the optimal model parameters for the configured machine learning classifier based on the repeated training and validation; selecting a second combination of hyperparameters from the matrix of candidate hyperparameter values and reconfiguring the machine learning classifier using the second combination of hyperparameters; performing the multiple iterations of training on the reconfigured machine learning classifier; repeating the selection of additional combinations of hyperparameters for training and validating until all combinations of hyperparameters are selected; and determining an optimal mode of the machine learning classifier with the optimal hyperparameters and the optimal model parameters that generate a best validation result.

Aspect 21. The method of any one of aspects 1 to 20, wherein the method further comprises deploying the optimal ARDS classifier for ARDS sub-phenotyping and personalized ARDS treatment.

Aspect 22. The method of aspect 21, wherein the deploying the optimal ARDS classifier for ARDS sub-phenotyping and providing personalized treatment comprises: hosting the optimal ARDS classifier on a cloud platform and exposing a plurality of application programming interfaces (APIs) of the optimal ARDS classifier, wherein the plurality of APIs comprises a set of input APIs for users to input ARDS patient data and a set of output APIs for outputting predicted ARDS sub-phenotype classifications.

Aspect 23. The method of any one of aspects 21 to 22, wherein the deploying the optimal ARDS classifier for ARDS sub-phenotyping and providing personalized treatment comprises: incorporating the optimal ARDS classifier into an Electronic Medical Record (EMR) system comprising a plurality of ARDS patient medical records; and displaying a user interface (UI) element for entering a plurality of clinical variables of the ARDS patient medical records into the optimal ARDS classifier and receiving an ARDS sub-phenotype classification result.

Aspect 24. The method of any one of aspects 21 to 23, wherein deploying comprises associating an ARDS patient with an ARDS sub-phenotype.

Aspect 25. The method of aspect 24, wherein the ARDS sub-phenotype is a one of two ARDS sub-phenotypes.

Aspect 26. The method of any one of 21 to 25, wherein the ARDS sub-phenotype is a Class 1 sub-phenotype associated with a high mortality risk or a Class 2 sub-phenotype associated with a low mortality risk.

Aspect 27. The method of any one of aspects 21 to 26, wherein the deploying the optimal ARDS classifier for ARDS sub-phenotyping and providing personalized ARDS treatment comprises: storing, in a database: (1) an ARDS sub-phenotype classification result generated by the optimal classifier based on the values of a plurality of clinical variables of the ARDS patient, and (2) explanatory information of the ARDS sub-phenotype classification result; and displaying a dashboard that visualizes (i) profile information of the ARDS patient, (ii) an ARDS sub-phenotype classification result for the ARDS patient, and (iii) a graphic user interface (GUI) comprising: (a) a first user interface (UI) element for providing access to the stored explanatory information, and (b) one or more additional UI elements for providing additional information on the ARDS sub-phenotype classification result.

Aspect 28. The method of any one of aspects 21 to 27, wherein before deploying the optimal ARDS classifier, the method further comprises: obtaining clinical data and physiological data from a plurality of randomized controlled ARDS clinical trial patient cohorts; inputting the physiological data into the optimal ARDS classifier to obtain a predicted classification result; and verifying the predicted classification result based on the clinical data to validate the optimal ARDS classifier.

Aspect 29. The method of aspect 28, wherein, the clinical data comprises age, gender, SAPS II score, SFA score, corticosteroids, Vaso_use_24h, vasopressor use, corticosteroid use, or a combination of any of the foregoing; and the physiological data comprises heart rate, temperature, mean arterial blood pressure, SPO₂, blood urea nitrogen, albumin, bilirubin, serum creatinine, whole blood creatinine, serum glucose, whole blood glucose, serum hematocrit, whole blood hematocrit, PCO₂ platelets, sodium, arterial TCO₂, venous TCO₂, white blood cell count, or a combination of any of the foregoing.

Aspect 30. The method of any one of aspects 21 to 29, wherein the deploying the optimal ARDS classifier for ARDS sub-phenotyping comprises: in response to input point-of-care variable values of an ARDS patient, generating a plurality of confidence scores respectively corresponding to a number of ARDS sub-phenotypes.

Aspect 31. The method of any one of aspects 1 to 30, further comprising: temporally tracking the values of the point-of-care variables of an ARDS patient and a series of ARDS sub-phenotype classification results generated by the optimal ARDS classifier for the ARDS patient; and generating an alert or notification when the tracked values of the point-of-care variables of the ARDS patient cause the optimal ARDS classifier to generate a new ARDS sub-phenotype classification result that is different from an immediately preceding ARDS sub-phenotype classification result.

Aspect 32. The method of aspect 31, wherein the notification comprises a recommended ARDS treatment.

Aspect 33. The method of any one of aspects 30 to 31, further comprising: temporally tracking gradients of the values of the point-of-care variables of the ARDS patient, wherein the ARDS patient is undergoing an ARDS treatment regimen; predicting future values of the point-of-care variables of the ARDS patient based on the tracked gradients; inputting the predicted future values of the point-of-care variables of the ARDS patient into the optimal ARDS classifier to predict a future ARDS sub-phenotype classification result for the ARDS patient; and generating an alert or notification when the predicted future ARDS sub-phenotype classification result is different than a current ARDS sub-phenotype classification result.

Aspect 34. The method of any one of aspects 1 to 33, the method further comprising: obtaining training data, wherein the training data comprises values for a plurality of clinical variables of the ARDS patient and additional information; and retraining the optimal ARDS classifier using the obtained training data and the additional information.

Aspect 35. The method of aspect 34, wherein the training data represent positive training data.

Aspect 36. The method of aspect 34, wherein the training data represent negative training data.

Aspect 37. The method of any one of aspects 34 to 36, wherein the obtaining training data comprises: constructing and executing a first Structured Query Language (SQL) query on a first database comprising ARDS patient records to identify ARDS patients with a first set of parameters for diagnosing ARDS; for the identified ARDS patients, constructing and executing a second SQL query on a second database comprising free-text report notes associated with ARDS patient candidate radiography imaging reports; executing a Natural Language Processing (NLP) model against the free-text report notes to identify ARDS patients with a second set of parameters that meet criteria consistent with a clinically recognized definition of ARDS, wherein the NLP model is trained to identify keywords with synonyms using supervised learning; and obtaining the ARDS patient monitoring records for the identified ARDS patients as the training data.

Aspect 38. The method of any one of aspects 1 to 37, wherein the clinically recognized definition of ARDS comprises the ARDS New Global Definition 2023.

Aspect 39. A system comprising one or more processors and one or more non-transitory computer-readable memories coupled to the one or more processors and configured with instructions executable by the one or more processors to cause the system to perform operations comprising: obtaining training data comprising ARDS patient records, wherein each patient record comprises a plurality of clinical variable values and clinical outcomes of historical ARDS patients; determining a number of ARDS sub-phenotypes based on the ARDS patient records using latent profile analysis; executing an iterative process for determining an optimal ARDS classifier for ARDS sub-phenotyping, wherein the iterative process comprises: constructing a plurality of machine learning classifiers, wherein the constructing comprises: configuring an output layer of each of the machine learning classifiers based on the number of ARDS sub-phenotypes; configuring an input layer of each of the plurality of machine learning classifiers to accept point-of-care variables in the plurality of clinical variables; and each machine learning classifier comprises a plurality of hyperparameters and a plurality of model parameters, for each machine learning classifier, training and validating the machine learning classifier using a grid search with multiple-fold cross-validation on the training data to determine an optimal mode of the machine learning classifier, wherein the point-of-care variable values in the training data are used as training input, and the clinical outcomes are used as training labels; and determining performance metrics for the optimal modes of the plurality of machine learning classifiers during the multiple-fold cross-validation; and identifying an optimal ARDS classifier from the optimal modes of the plurality of machine learning classifiers by comparing the performance metrics.

Aspect 40. The system of aspect 39, wherein the system is further configured to deploy the ARDS optimal classifier for ARDS sub-phenotyping and providing personalized treatment to the ARDS patient based on the optimal classifier.

Aspect 41. A non-transitory computer-readable storage medium configured with instructions executable by one or more processors to cause the one or more processors to perform operations comprising: obtaining training data comprising ARDS patient records, wherein each ARDS patient record comprises a plurality of clinical variables and clinical outcomes of historical patients; determining a number of ARDS patient sub-phenotypes based on the ARDS patient records using latent profile analysis; executing an iterative process for determining an optimal ARDS classifier for ARDS patient sub-phenotyping, wherein the iterative process comprises: constructing a plurality of machine learning classifiers, wherein the constructing comprises: configuring an output layer of each of the plurality of machine learning classifiers based on the number of ARDS patient sub-phenotypes, configuring an input layer of each of the plurality of machine learning classifiers to accept point-of-care variables in the plurality of clinical variables, and each of the plurality of machine learning classifiers comprises a plurality of hyperparameters and a plurality of model parameters, for each of the plurality of machine learning classifiers, training and validating the machine learning classifier using a grid search with multiple-fold cross-validation on the training data to determine an optimal mode of the machine learning classifier, wherein the point-of-care variables in the training data are used as training input, and the clinical outcomes are used as training labels, and determining performance metrics for the optimal modes of the plurality of machine learning classifiers during the multiple-fold cross-validation; and identifying the optimal ARDS classifier from the optimal modes of the plurality of machine learning classifiers by comparing the performance metrics.

Aspect 42. The non-transitory computer-readable storage medium of aspect 41, wherein the non-transitory computer-readable storage medium is further configured to deploy the optimal ARDS classifier for ARDS sub-phenotyping and providing personalized treatment to the ARDS patient based on the optimal ARDS classifier.

Aspect 43. A method for ARDS sub-phenotyping, comprising: obtaining clinical data of an ARDS patient over a duration, wherein the clinical data comprises point-of-care variable values, and excludes variables associated with biomarkers and ventilator-based parameters; entering the obtained clinical data into a classifier trained for ARDS sub-phenotyping to generate an ARDS sub-phenotype classification result, wherein the ARDS sub-phenotype classification result comprises one of a plurality of ARDS sub-phenotypes; storing the obtained clinical data of the ARDS patient and the ARDS sub-phenotype classification result into an Electronic Medical Record (EMR) system; temporally tracking the obtained clinical and the ARDS sub-phenotype classification result; and generating an alert or notification when a new ARDS sub-phenotype classification result is different than a previous ARDS sub-phenotype classification result.

Aspect 44. An optimal ARDS classifier determined according to the method of any one of aspects 1 to 38.

Aspect 45. An ARDS sub-phenotype classification of an ARDS patient determined using the optimal ARDS classifier of aspect 44.

Aspect 46. A method of treating the ARDS patient based on the ARDS sub-phenotype classification of aspect 45.

Aspect 47. The method of any one of aspects 45 to 46 wherein the ARDS sub-phenotype is one of a Class 1 ARDS sub-phenotype associated with a higher mortality risk and a Class 2 ARDS sub-phenotype associated with a higher mortality risk.

Aspect 48. The method of aspect 47, wherein, the ARDS patient represents a Class 1 ARDS sub-phenotype; and the method comprises administering a treatment to the ARDS patient suitable for treating the Class 1 ARDS sub-phenotype.

Aspect 49. The method of aspect 47, wherein, the ARDS patient represents a Class 2 ARDS sub-phenotype; and the method comprises administering a treatment to the ARDS patient suitable for treating the Class 2 ARDS sub-phenotype.

Aspect 50. The method of aspect 47, wherein, the ARDS patient is identified as having a Class 1 ARDS phenotype; and treating comprises administering neuromuscular blockade (NMB) therapy or no NMB therapy, high PEEP or low PEEP, no treatment or methylprednisolone, dexamethasone, no lisofylline, ketoconazole, catheter and fluid treatment, recruitment maneuver, statins, full or trophic enteral feeding, or a combination of any of the foregoing.

Aspect 51. The method of aspect 47, wherein, the ARDS patient is identified as having a Class 2 ARDS phenotype; and treating comprises administering NMB therapy, low PEEP therapy, no methylprednisolone, no treatment or dexamethasone, no treatment or lisofylline, no treatment or ketoconazole, no combination of catheter and fluid treatment, no recruitment maneuver, statins as a preemptive therapy, full enteral feeding, or a combination of any of the foregoing.

Aspect 52. The method of aspect 47, wherein treating comprises administering an intervention suitable for treating the determined ARDS phenotype of the ARDS patient, wherein the intervention is selected from airway pressure release ventilation, continuous positive airway pressure, driving pressure, extracorporeal membrane oxygenation, extracorporeal carbon dioxide removal, fluid management, glucocorticoid administration, high-flow nasal oxygen, high-frequency oscillatory ventilation, inhaled pulmonary vasodilators, low tidal volume ventilation, lung-protective ventilation, mechanical power adjustment, neuromuscular blocking agents, plateau pressure limitation, positive end expiratory pressure (peep) titration, prone positioning, recruitment maneuvers, systemic corticosteroids, tidal volume limitation, veno-venous extracorporeal membrane oxygenation, ventilatory support, or a combination of any of the foregoing.

Aspect 53. A method of generating an ARDS sub-phenotype trajectory map of an ARDS patient comprising: generating a first ARDS sub-phenotype classification result based on a first set of point-of-care clinical data for the ARDS patient; generating a second ARDS sub-phenotype classification result based on a second set of point-of-care clinical data for the ARDS patient; and generating an ARDS sub-phenotype classification trajectory map based on the first ARDS sub-phenotype classification result and the second ARDS sub-phenotype classification result.

Aspect 54. The method of aspect 53, wherein the method comprises: providing the first set of point-of-care clinical data of an ARDS patient obtained at a first time; and providing the second set of point-of-care clinical data of an ARDS patient obtained at a second time.

Aspect 55. The method of aspect 54, wherein, the first set of point-of-care clinical data comprises values for each point-of-care clinical variable of a set of a first point-of-care clinical variables; and the second set of point-of-care clinical data comprises values for each point-of-care clinical variable of a second set of point-of-care clinical variables.

Aspect 56. A method of generating an ARDS sub-phenotype trajectory map of an ARDS patient comprising: providing a longitudinal dataset comprising values for each point-of-care clinical variable of a set of point-of-care clinical variables of an ARDS patient obtained at two or more times; generating an ARDS sub-phenotype classification result based on the longitudinal dataset; and generating an ARDS sub-phenotype classification trajectory map based on the ARDS sub-phenotype classification result corresponding to each of the two or more times.

Aspect 57. The method of any one of aspects 53 to 56, wherein the set of point-of-care clinical data does not include values for biomarker variables and values for non-ventilator variables.

Aspect 58. The computer-implemented method of any one of aspects 53 to 57, wherein the set of point-of-care clinical data comprises a plurality of physiological parameters.

Aspect 59. The method of aspect 58, wherein the plurality of physiological parameters comprises platelet concentration, SpO₂, serum creatinine concentration, serum hematocrit value, white blood cell count, sodium concentration, heart rate, serum glucose concentration, or a combination of any of the foregoing.

Aspect 60. The method of aspect 58, wherein the plurality of physiological parameters comprises platelet concentration, SpO₂, serum creatinine concentration, serum hematocrit value, white blood cell count, sodium concentration, heart rate, and serum glucose concentration.

Aspect 61. The method of aspect 58, wherein the plurality of physiological parameters consists of platelet concentration, SpO₂, serum creatinine concentration, serum hematocrit value, white blood cell count, sodium concentration, heart rate, and serum glucose concentration.

Aspect 62. The method of aspect 56, wherein the longitudinal dataset comprises point-of-care clinical data obtained at from 2 to 20 different times.

Aspect 63. The method of any one of aspects 56 and 62, wherein the longitudinal dataset comprises point-of-care clinical data obtained during a period of from 1 hour to 12 hours.

Aspect 64. The method of any one of aspects 1 to 11, wherein generating an ARDS sub-phenotype classification result comprises: providing an optimal ARDS classifier for ARDS sub-phenotyping; and inputting the point-of-care clinical data into the optimal ARDS classifier to obtain a ARDS sub-phenotype classification result.

Aspect 65. The method of any one of aspects 53 to 64, wherein the optimal ARDS classifier is determined according to the method of any one of aspects 1 to 38.

Aspect 66. The method of any one of aspects 53 to 65, wherein generating an ARDS sub-phenotype classification result comprises applying the optimal ARDS classifier of aspect 1 to point-of-care clinical data of a patient.

Aspect 67. The method of any one of aspects 53 to 66, wherein the method further comprises predicting a future ARDS sub-phenotype classification result of the ARDS patient based on the ARDS sub-phenotype classification trajectory map.

Aspect 68. The method of any one of aspects 53 to 67, wherein the method further comprises predicting whether the ARDS patient will exhibit a different ARDS sub-phenotype classification result at a future time based on the ARDS sub-phenotype classification trajectory map.

Aspect 69. The method of any one of aspects 53 to 68, wherein the method further comprises predicting when in the future the ARDS patient will exhibit a different ARDS sub-phenotype classification result based on the ARDS sub-phenotype classification trajectory map.

Aspect 70. The method of any one of aspects 53 to 69, wherein the method comprises recommending an ARDS treatment based on the ARDS sub-phenotype trajectory map.

Aspect 71. An ARDS sub-phenotype trajectory map for an ARDS patient generated using the method of any one of aspects 53 to 70.

Aspect 72. A method of treating ARDS in a patient comprising administering an ARDS treatment to the ARDS patient based on the ARDS sub-phenotype trajectory map of any one of aspects 53 to 71.

Aspect 73. A system comprising one or more processors and one or more non-transitory computer-readable memories coupled to the one or more processors and configured with instructions executable by the one or more processors comprising: an ARDS sub-phenotype classification module; an ARDS sub-phenotype trajectory mapping module; and an ARDS clinical decision support module.

Aspect 74. The system of aspect 73, wherein the ARDS sub-phenotype classification module is configured to generate an ARDS sub-phenotype classification result based on an optimal ARDS classifier and two or more sets of point-of-care clinical data for an ARDS patient.

Aspect 75. The system of aspect 74, wherein the optimal ARDS classifier is determined according to any one of aspects 1 to 38.

Aspect 76. The system of any one of aspects 73 to 75, wherein the ARDS sub-phenotype trajectory mapping module is configured to generate an ARDS sub-phenotype trajectory map based on longitudinal ARDS sub-phenotype classification results of an ARDS patient.

Aspect 77. The system of any one of aspects 73 to 76, wherein the ARDS clinical decision support module is configured to provide a treatment recommendation for the ARDS patient based on an ARDS sub-phenotype trajectory map of an ARDS patient.

Aspect 78. A non-transitory computer-readable storage medium configured with instructions executable by one or more processors to cause the one or more processors to perform operations comprising: generating a first ARDS sub-phenotype classification result based on a first set of point-of-care clinical data for the ARDS patient; generating a second ARDS sub-phenotype classification result based on a second set of point-of-care clinical data for the ARDS patient; and generating an ARDS sub-phenotype classification trajectory map based on the first ARDS sub-phenotype classification result and the second ARDS sub-phenotype classification result.

Aspect 79. The non-transitory computer-readable storage medium configured of aspect 26, wherein generating an ARDS sub-phenotype classification result comprises: providing an optimal ARDS classifier for ARDS sub-phenotyping; and inputting the point-of-care clinical data into the optimal ARDS classifier to obtain a ARDS sub-phenotype classification result.

Aspect 80. The non-transitory computer-readable storage medium configured of any one of aspects 26 and 27, wherein the optimal ARDS classifier is determined according to the method of any one of aspects 1 to 38.

Aspect 81. The non-transitory computer-readable storage medium of any one of aspects 26 and 27, wherein obtaining ARDS sub-phenotype classification result comprises applying the optimal ARDS classifier of aspect 44.

Aspect 82. A method of determining an ARDS sub-phenotype of an ARDS patient comprising: inputting a plurality of clinical variables into the optimal ARDS classifier of aspect 44; and determining an ARDS sub-phenotype classification result of the ARDS patient.

Aspect 83. The method of aspect 82, wherein the method comprises, before inputting the plurality of clinical variables, determining the values for the plurality of clinical variables of the ARDS patient.

Aspect 84. The method of any one of aspects 82 to 83, wherein the plurality of clinical variables comprises point-of-care variables.

Aspect 85. The method of any one of aspects 82 to 83, wherein the plurality of clinical variables comprises heart rate, temperature, mean arterial blood pressure, SpO₂, blood urea nitrogen, albumin, bilirubin, serum creatinine, whole blood creatinine, serum glucose, whole blood glucose, serum hematocrit, whole blood hematocrit, PCO₂ platelets, sodium, arterial TCO₂, venous TCO₂, white blood cell count, or a combination of any of the foregoing.

Aspect 86. The method of any one of aspects 82 to 83, wherein the plurality of clinical variables comprises heart rate, SpO₂, serum creatinine, serum glucose, serum hematocrit, platelets, sodium, and white blood cell count.

Aspect 87. The method of any one of aspects 82 to 83, wherein the plurality of clinical variables consists of point-of-care variables.

Aspect 88. The method of any one of aspects 82 to 83, wherein the plurality of clinical variables consists of heart rate, temperature, mean arterial blood pressure, SpO₂, blood urea nitrogen, albumin, bilirubin, serum creatinine, whole blood creatinine, serum glucose, whole blood glucose, serum hematocrit, whole blood hematocrit, PCO₂ platelets, sodium, arterial TCO₂, venous TCO₂, white blood cell count, or a combination of any of the foregoing.

Aspect 89. The method of any one of aspects 82 to 83, wherein the plurality of clinical variables consists of heart rate, SpO₂, serum creatinine, serum glucose, serum hematocrit, platelets, sodium, and white blood cell count.

Aspect 90. The method of any one of aspects 82 to 89, wherein the ARDS sub-phenotype classification result is one of a Class 1 ARDS sub-phenotype associated with a high mortality risk and a Class 2 ARDS sub-phenotype associated with a low mortality.

Although an overview of the subject matter has been described with reference to specific examples, various modifications and changes may be made to the examples without departing from the broader scope of embodiments of the present disclosure.

### EXAMPLES

Embodiments provided by the present disclosure are further illustrated by reference to the following examples, which describe certain details of methods and systems for ARDS patient sub-phenotyping provided by the present disclosure.

### Example 1

### Endotyping ARDS Patients

The goal of this study was to establish a method to endotype ARDS patients consistent with the New Global Definition 2023 into clinically relevant ARDS sub-phenotypes using readily accessible (non-biomarker and non-ventilator) clinical variables.

Data from the MIMIC-IV Version 2.2 Database https://physionet.org/content/mimiciv/2.2/) were retrospectively analyzed in the study. The database includes ICU-monitoring data from patients in the ICU at Beth Israel Deaconess Medical Center between 2008 and 2019. This data was used to create and train the model for classifying ARDS patients into sub-phenotypes. The final model was tested and validated using clinical and biological data from five (5) independent datasets obtained from patients enrolled in the NHLBI ARDS Network's Randomized Controlled Trials including ARMA, ALVEOLI, FACTT, EDEN, and SAILS.

Of the 299,712 unique patients listed in the MIMIC-IV Database, only patients above the age of 18 that met the ARDS criteria using the New Global Definition 2023 were included in the study. No patients were excluded from the Randomized Controlled Trials.

Structured Query Language (SQL) software (IBM Corp., Armonk, NY, USA) was used to extract data from the MIMIC-IV Database. All data used from the MIMIC-IV Database were collected at the time of first admission into an ICU. All clinical data and biological data (other than outcomes) from the Randomized Controlled Trials used for this analysis were collected at study baseline (pre-randomization). The primary outcome in this study was 30-day mortality after admission to the hospital.

Feature selection was based on previous research and enabled the classification of non-biomarker and non-ventilator patients according to the New Global Definition 2023 of ARDS. The following clinical variables were used in the study: heart rate, temperature, mean arterial blood pressure, SPO₂, mean non-invasive blood pressure, hematocrit (whole blood), hematocrit (serum), WBCs, platelets, sodium, creatinine (whole blood), creatinine (serum), glucose (whole blood), glucose (serum), albumin, bilirubin, blood urea nitrogen (BUN), total CO₂ arterial, total CO₂ venous, and PCO₂. The baseline values for these variables were characterized as the first recorded value within 24 hours of admission into ICU.

During the data pre-processing phase, outliers with values beyond the 1st and 99th percentiles were identified and replaced with null values. Variables with over 10% missing data were excluded from the analysis. For variables with less than 10% missing data, multiple imputation using the chained equation (MICE) approach was employed. To enhance the accuracy of the subsequent models, all non-normally distributed variables underwent log-transformation. The variables were standardized to a zero mean and unit standard deviation. Collinearity analysis was conducted to identify and address variables with high intercorrelations, ensuring data integrity and minimizing redundancy. Following these steps, the variables were prepared for Latent Profile Analysis (LPA).

Latent Profile Analysis (LPA) was done using curated data from the time of first admission to the ICU to identify patient sub-phenotypes. The optimal number of latent classes was selected using the lowest Bayesian information criterion and integrated complete likelihood values. Entropy and probability of group membership were used to evaluate class separation and robustness of class membership. The clusters found were also analyzed to understand their clinical and biological differences, and any results which did not show significance were discarded.

The next step was to use a supervised learning algorithm to prospectively identify patient cluster membership using the minimum number of readily available clinical variables. Five (5) different machine learning algorithms were trained on the clustering dataset: Logistic Regression, Random Forest, Support Vector Machine (SVM), LightGBM and XGBoost. SVM was able to classify ARDS patients into the identified clusters with the most accuracy. Hyperparameter tuning was performed by running a grid search with 5-fold cross-validation. The “best model” was selected using metrics of accuracy score, f-1 score, and ROC-AUC on each of the 5 cv folds.

The SVM model was tested using the Randomized Controlled Trials. The performance of the model on the Randomized Controlled Trial cohorts was assessed by comparing the model baseline characteristics and patient clinical outcomes segmented by class membership. The levels of biomarkers were also compared between patients assigned to each class for the ALVEOLI cohort.

The Wilcoxon rank-sum test was used to compare baseline characteristics between the two sub-phenotype classes. The chi-square test was used to compare the clinical outcome variables such as mortality (i.e., proportion of deaths) between the sub-phenotype classes. The log-rank test was used to determine differences in survival curves. p-Values less than 0.01 were considered statistically significant.

Using the data from the RCT cohorts, contingency analysis was performed to assess whether a heterogeneity of treatment effect (HTE) on mortality based on the ARDS sub-phenotype classes could be identified for the interventions evaluated. As detailed in the supplement, we considered interaction with (1) pulmonary artery catheter (PAC) vs. central venous catheter (CVC) and liberal vs. conservative fluid management in the FACTT trial on 90-day mortality; (2) initial trophic enteral feeding vs. early advancement to full-calorie enteral feeding in the EDEN trial on 90 day mortality; (3) rosuvastatin vs. placebo in the SAILS trial on 90 day mortality; (4) high vs. low tidal volume in the ARMA trial on 180-day mortality; and (5) higher vs. lower PEEP treatments in the ALVEOLI trial on 90-day mortality.

There were 299,712 unique listed patients in the MIMIC-IV Database. Of these, 1,417 patients met the criteria for ARDS using the New Global Definition 2023. ARDS patients had a mean age of 62 years, median height of 170 cm, and mean weight of 83 kg.

Latent Profile Analysis (LPA) was used to classify the ARDS patients. Bayesian Information Criterion (BIC), Bootstrap Likelihood Ratio Test (BLRT), and Integrated Complete Likelihood (ICL) method were used to determine the optimal number of patient clusters.

Using Bayesian Information Criterion (BIC), diminishing improvements upon increasing cluster sizes beyond 2 was observed. This trend was further supported using Bootstrap Likelihood Ratio Test (BLRT) values. Although probability values (p-values) for BLRT remained below 0.01, BLRT values reduced as the number of classes increased beyond 2, suggesting diminishing model improvement with the introduction of additional classes. Although ICL values were highest for the 4-class model, class sizes were considerably imbalanced. The 2-class solution exhibited a more equitable distribution of subjects across classes (28.79% and 71.21%).

A comparison between the classes and SOFA score was conducted. The mean SOFA score was 5.72, of which 133 out of 408 patients (32.60%) with SOFA<5.72 were in Class 1, and 395 out of 1006 patients (39.26%) with SOFA>5.72 were in Class 2. Contingency analysis using McNemar's test showed a significant statistical difference between the classes and patient groups defined using SOFA score with p-value<0.0001. Additionally, the 30-day mortality rate difference between the clusters determined by the SOFA score classification and the clusters determined by our ARDS model was compared. Greater differences in clinical outcomes between the calculated ARDS sub-phenotypes (38.7% vs. 22.8%; 15.7% difference in mortality rates) than those defined based on categorization by the mean SOFA score (32.1% above the mean vs. 23.3% below the mean; 8.8% difference in mortality rates) were observed.

Latent Profile Analysis (LPA) was used to classify the patients into two distinct classes. There were 408 patients (28.79%) in Class 1 and 1,009 patients (71.21%) in Class 2. There was a statistically significant difference (with p<0.01) between Class 1 and Class 2 in the values for platelet counts, SPO₂ (%), serum hematocrit, white blood cells count, sodium, serum glucose, serum creatinine and heart rate. Class 1 had a lower median value for serum glucose than Class 2, but the difference was not statistically significant (p=0.15). FIGS. 6A-6C show the distribution of the patients within each class across all variables in the form of a box-and-whisker plots.

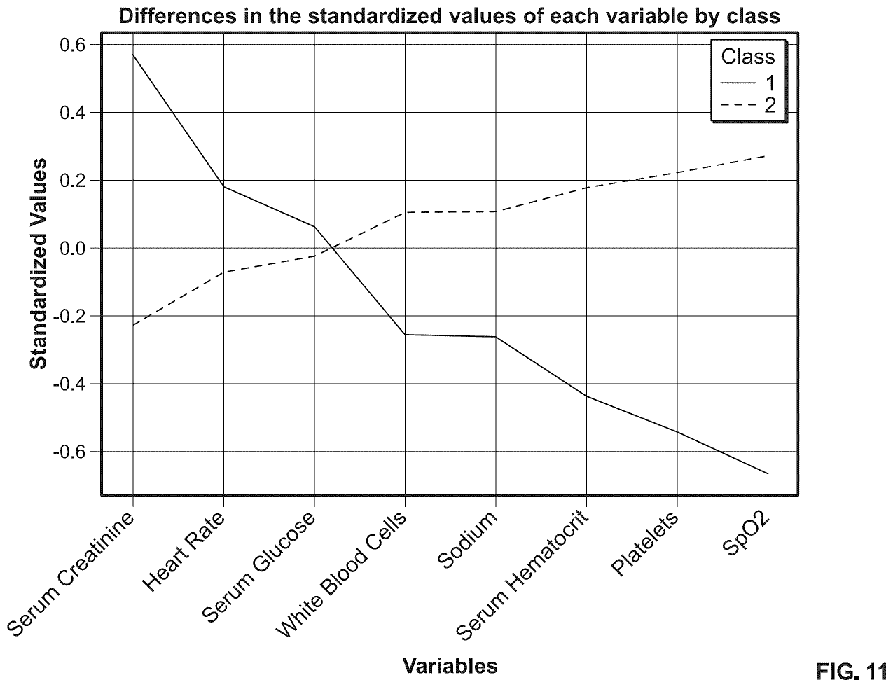

The differences between the classes using mean standardized values of each variable are shown in FIG. 11. Class 1 had higher mean standardized values for SpO₂ (%), platelet counts, serum hematocrit, white blood cells count, and sodium than Class 2, whereas Class 1 had lower mean standardized values for serum creatinine, heart rate, and serum glucose.

The association between class assignment and clinical outcomes was analyzed to determine whether the classes had different progression patterns. The clinical outcomes by class are summarized in Table 1. Patients in Class 1 had a significantly higher 30-day mortality rate compared to patients in Class 2 (38.73% vs 22.79%; p<0.01). This difference extended to 90-day mortality rates (50.00% vs 31.52%; p<0.01). The in-hospital mortality rate was also higher for patients in Class 1 compared to those in Class 2 (41.42% vs 23.89%; p<0.01).

TABLE 1
Clinical outcomes by class.

| Outcome Variable | Class 1 (n = 408) | Class 2 (n = 1009) | p-value |
|---|---|---|---|
| 30-day Mortality | 158 (38.7%) | 230 (22.8%) | <0.0001 |
| 90-day Mortality | 204 (50.0%) | 318 (31.5%) | <0.0001 |
| In-hospital Mortality | 169 (41.4%) | 241 (23.9%) | <0.0001 |

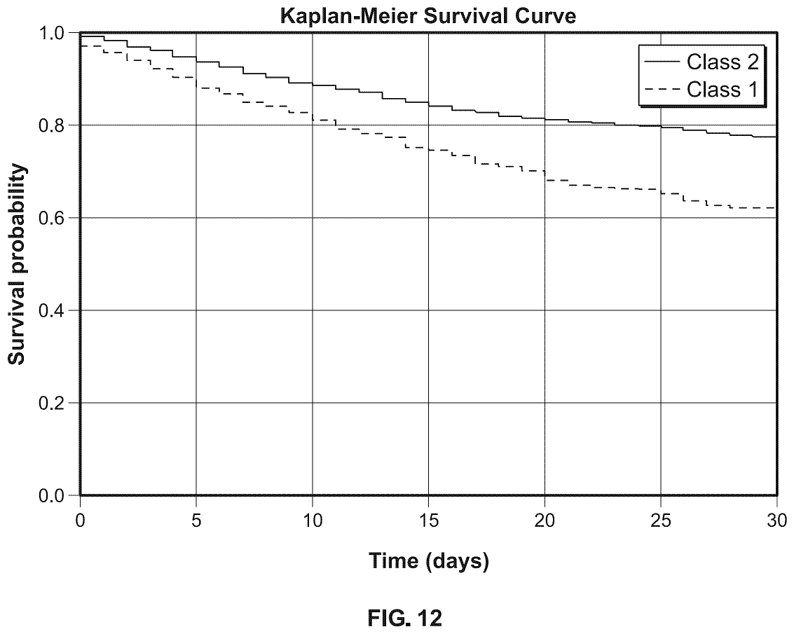

A Kaplan Meier survival curve was used to visualize the survival probabilities of different ARDS classes over time. The Kaplan Meier survival curve for the patients from each class at day 30 is provided in FIG. 12. Patients in Class 1 had a significantly lower survival probability than the patients in Class 2 (p<0.0001).

Five (5) different supervised machine learning models were evaluated to predict patient class membership. The optimal estimators for each model were selected using grid search with 5-fold cross validation. Models trained using respective optimal parameters were compared against each other using metrics of accuracy, f1 score, and ROC-AUC. Of the five models, SVM had the highest mean (Accuracy=96.47%, f-1 Score=97.51%, and ROC-AUC=99.45%) and lowest standard deviation across all metrics. The SVM model was determined to be the best model for prospectively classifying patients into the two ARDS classes determined by the LPA analysis. FIG. 6C shows a comparison of ROC-AUC performance of the five models.

The best performing SVM model was run on an independent test set (ALVEOLI Cohort) to evaluate model performance on an independent patient population. The SVM model classified 136 (24.73%) ALVEOLI study participants into Class 1 and the remaining 414 participants (75.27%) into Class 2. As shown in Table 2, all clinical variables showed consistent differences as before in the MIMIC-IV dataset. The differences were found to be statistically significant (with a p<0.01) in almost all variables.

TABLE 2
Differences in clinical variables between predicted patient classes.

| | Class 1 | Class 2 | |
|---|---|---|---|
| Variable | (n = 136) | (n = 414) | p-value |
| SpO₂ (%) | ¹ 93.0 (90.0-95.0) | 96.0 (94.0-98.0) | <0.0001 |
| Platelets | 80.0 (40.0-175.0) | 170.0 (107.0-250.0) | <0.0001 |
| (10⁹/L) | | | |
| Creatinine | 1.7 (0.9-3.1) | 1.0 (0.8-1.6) | <0.0001 |
| (mg/dL) | | | |
| Hematocrit (%) | 27.5 (24.0-31.0) | 30.0 (27.0-34.0) | <0.0001 |
| Heart Rate | 132.0 (115.0-149.0) | 123.0 (105.0-136.0) | <0.0001 |
| (bpm) | | | |
| White Blood | 12.0 (6.1-17.2) | 13.2 (9.4-18.3) | 0.0037 |
| Cells (10⁹/L) | | | |
| Sodium | 136.0 (132.0-139.0) | 137.0 (134.0-140.0) | 0.0159 |
| (mmol/L) | | | |
| Glucose | 111.5 (93.0-150.2) | 119.0 (97.0-150.0) | 0.2845 |
| (mg/dL) | | | |

¹ Mean (min-max)

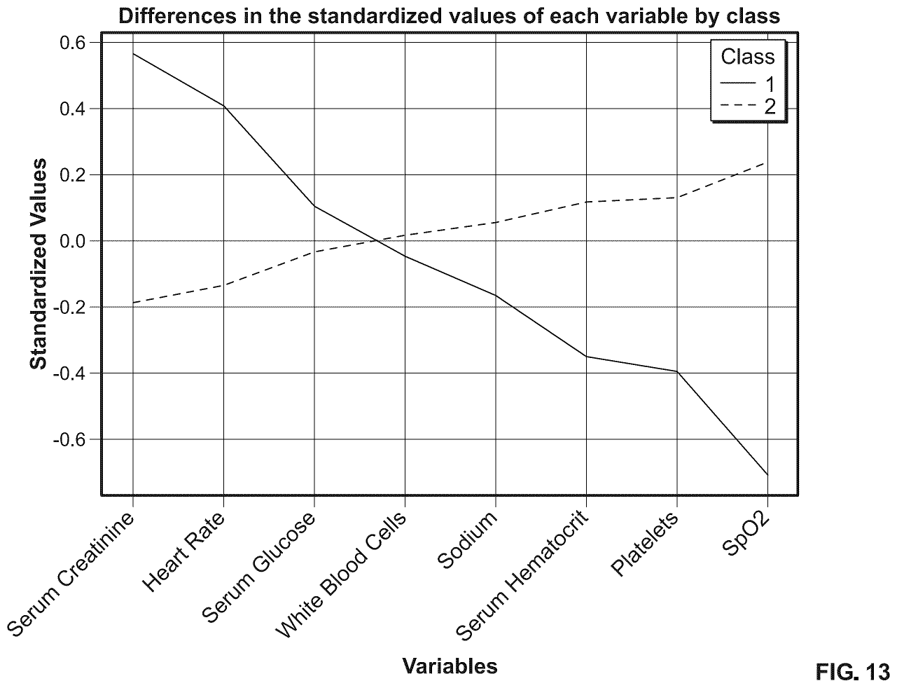

For comparison with the training set, the differences in the means of the standardized values of each variable by class for ALVEOLI dataset is shown in FIG. 13. The direction of these differences was similar to that of the LPA classes previously defined in the MIMIC-IV Database (FIG. 11).

The best performing SVM model was subsequently run on all Randomized Controlled Trial datasets: ARMA, ALVEOLI, FACTT, EDEN, and SAILS. The 30-day mortality rate of the predicted classes for each dataset followed a similar trend as observed in the MIMIC-IV Database using the LPA classes. As shown in Table 3, ARDS patients in Class 1 had a significantly higher mortality rate than those in Class 2.

TABLE 3
30-day mortality rates of predicted patient classes by RCT.

| Study | Class 1 Mortality | Class 2 Mortality | p-value |
|---|---|---|---|
| ARMA (n = 667) | 169 (45.7%) | 498 (32.3%) | 0.0007 |
| ALVEOLI (n = 550) | 136 (43.4%) | 414 (21.8%) | <0.0001 |
| FACTT (n = 1000) | 233 (41.4%) | 767 (25.2%) | <0.0001 |
| EDEN (n = 1000) | 214 (31.0%) | 786 (21.5%) | 0.0037 |
| SAILS (n = 745) | 199 (39.9%) | 546 (23.2%) | <0.0001 |

To highlight the biological significance of the two predicted patient classes, biomarker levels were analyzed for each class using the ALVEOLI dataset which contained biomarker values for interleukin 6 (IL-6), intercellular adhesion molecule 1 (ICAM-1), and surfactant protein D (SPD). The summary of this analysis is shown in Table 4. The median values of all three biomarkers were found to be consistently higher for patients in Class 1 than those in Class 2. The difference was statistically significant (p<0.01) for IL-6 and ICAM-1.

TABLE 4
Biomarker differences between predicted patient classes.

| Biomarker | Class 1 (n = 136) | Class 2 (n = 414) | p-value |
|---|---|---|---|
| IL-6 (pg/mL) | 358 (127-2341) | 201 (89-547) | 0.000159 |
| ICAM-1 (pg/mL) | 1060 (755-1588) | 885 (587-1275) | 0.0007 |
| SPD (pg/mL) | 116 (49-277) | 98 (50-199) | 0.3457 |

The association between class assignment and heterogeneity of treatment effect on mortality within the five ARDSnet RCT cohorts was assessed. In the FACTT dataset, the effect of the interaction between catheter strategy (PAC or CVC) and the predicted ARDS classes was not statistically significant on 90-day mortality (p-value>0.44). Interaction between fluid management (liberal vs. conservative) in the FACTT trial and predicted ARDS classes was also not significant (p-value>0.62) on 90-day mortality. In the EDEN dataset, interaction between feeding strategies (initial trophic vs. full-calorie) and predicted ARDS classes was not significant on 90-day mortality (p-value>0.2). In the SAILS dataset, interaction between treatment (rosuvastatin vs. placebo) and predicted ARDS classes was not significant on 90-day mortality (p-value>0.74). In the ARMA dataset, interaction between tidal volume (high vs. low) and predicted ARDS classes was not significant on 180-day mortality (p-value>0.56). In the ALVEOLI dataset, the interaction between the ventilator strategy (high or low PEEP) and the predicted ARDS classes was not statistically significant on 90-day mortality (p-value>0.51). This suggests an absence of a differential treatment effect in the RCT datasets based on the sub-phenotypes predicted by the present model on longer-term mortality.

### Example 2

### Comparison to SOFA Score

To understand the specificity of the model, the model sub-phenotypes were compared with those defined by non-specific disease severity scores such as SOFA. McNemar's test on the contingency table yielded a statistically significant result, highlighting that the model sub-phenotypes were very distinct from those defined using SOFA scores. Greater differences in clinical outcomes between the determined ARDS sub-phenotypes than those defined using SOFA were observed. These results suggest that the present model separates patients in the specific context of ARDS, rather than just identify patients who are more ill. Furthermore, although the present model only uses objective inputs, many disease severity scores (including SOFA and APACHE) use subjective measurements such as the Glasgow Coma Scale (GCS) in the calculations.

In the analysis, the relationship between class assignments and the Sequential Organ Failure Assessment (SOFA) score among patients with ARDS in the MIMIC database was evaluated. This involved comparing the baseline SOFA scores within each class to understand the clinical severity associated with each group.

To statistically test the independence of class distribution between the ARDS sub-phenotype model and the SOFA score classification, McNemar's test was conducted using the ‘contingency_tables’ module from the ‘statsmodels.stats’ library in Python.

Table 5 shows the classification matrix between SOFA Model with 5.72 threshold (mean of SOFA score for all ARDS patients in MIMIC-IV) and LPA Classification Model:

TABLE 5
Classification Matrix.

| | | SOFA Cluster (threshold 5.72) | |
|---|---|---|---|
| | | Class 1 | Class 2 |
| | | (Higher | (Lower |
| | | Mortality | Mortality |
| | | Risk) | Risk) |
| ARDS Model | Class 1 (Higher Risk) | 275 | 133 |
| Cluster | Class 2 (Lower Risk) | 395 | 611 |

The p-value with continuity correction for this contingency table using McNemar's test is <0.0001, suggesting extremely statistically significant differences between the two classifications.

The present model can classify ARDS patients into two distinct ARDS sub-phenotypes with 99% accuracy in the validation cohort. The ARDS sub-phenotypes show distinct mortality rates and other characteristics, consistent with previously identified hypo- and hyper-inflammatory ARDS sub-phenotypes. The ARDS sub-phenotypes show high disease specificity, which is distinct from classification using SOFA score (p<0.0001), and with no Heterogeneity of Treatment Effect (HTE) in the RCTs (p>0.2).

In summary, a simplified ML model was developed to prospectively and to accurately identify ARDS sub-phenotypes using only eight readily available clinical variables. These ARDS sub-phenotypes displayed greater differences in mortality rates than observed when using SOFA score-based categorization.

The embodiments illustrated herein are described in sufficient detail to enable those skilled in the art to practice the teachings disclosed. Other embodiments may be used and derived based on the present disclosure and structural and logical substitutions and changes may be made without departing from the scope of this disclosure. Accordingly, the present disclosure is to be considered as illustrative and not restrictive. Furthermore, the claims are not to be limited to the present disclosure and are entitled their full scope and equivalents.

What is claimed is:

1. A method for determining an optimal acute respiratory distress syndrome (ARDS) classifier for ARDS sub-phenotyping, comprising:
obtaining training data comprising ARDS patient records, wherein each ARDS patient record comprises a plurality of clinical variables and a plurality of clinical outcomes;
determining a number of ARDS sub-phenotypes based on the ARDS patient records using latent profile analysis;
executing an iterative process to determine an optimal ARDS classifier for determining the number of ARDS sub-phenotypes, wherein the iterative process comprises:
constructing a plurality of machine learning classifiers, wherein,
the constructing comprises:
configuring an output layer of each of a plurality of machine learning classifiers based on the number of ARDS sub-phenotypes;
configuring an input layer of each of the plurality of machine learning classifiers to accept the plurality of clinical variables; and
each of the machine learning classifiers independently comprises a plurality of hyperparameters and a plurality of model parameters;
for each of the machine learning classifiers, training and validating the machine learning classifier using a grid search with multiple-fold cross-validation on the training data to determine an optimal mode of the machine learning classifier, wherein the plurality of clinical variables of the training data are used as training input, and the clinical outcomes of the training data are used as training labels; and
determining performance metrics for the optimal mode of each machine learning classifier during the multiple-fold cross-validation; and
identifying the optimal ARDS classifier based on the optimal modes for each machine learning classifier of the plurality of machine learning classifiers by comparing the performance metrics.

2. The method of claim 1, wherein the number of ARDS sub-phenotypes is two.

3. The method of claim 1, wherein the plurality of clinical variables does not include clinical variables associated with biomarkers and clinical variables associated with ventilator-based parameters.

4. The method of claim 1, wherein the plurality of clinical variables comprises a plurality of point-of-care clinical variables.

5. The method of claim 4, wherein the point-of-care clinical variables consist of platelet concentration, SpO₂, serum creatinine concentration, serum hematocrit value, white blood cell count, sodium concentration, heart rate, and serum glucose concentration.

6. The method of claim 4, wherein the plurality of clinical outcomes comprise mortality data.

7. The method of claim 1, wherein the optimal mode of the machine learning classifier comprises optimal hyperparameters and optimal model parameters of the machine learning classifier, wherein,
the optimal hyperparameters are used to configure the training of the machine learning classifier; and
the optimal model parameters are adjusted during the training of the optimal model parameters.

8. The method of claim 1, wherein the performance metrics comprise accuracy, F1 score, ROC-AUC (Receiver Operating Characteristic-Area Under the Curve), or a combination of any of the foregoing.

9. The method of claim 1, wherein identifying the optimal ARDS classifier comprises selecting the machine learning classifier of the plurality of machine learning classifiers having the highest ROC-AUC compared to the other of the plurality of machine learning classifiers.

10. The method of claim 1, wherein the plurality of machine learning classifiers comprises a Light Gradient Boosting Machine algorithm, a Logistic Regression algorithm, a Random Forest algorithm, a Support Vector Machine algorithm, an Extreme Gradient Boosting algorithm, or a combination of any of the foregoing.

11. The method of claim 1, wherein the optimal ARDS classifier is a Support Vector Machine algorithm.

12. The method of claim 1, before determining the number of ARDS sub-phenotypes, performing data cleaning and data preprocessing on the ARDS patient records to obtain the training data.

13. The method of claim 1, wherein the training and validating the machine learning classifier using a grid search with multiple-fold cross-validation on the training data comprises:
creating a matrix of candidate hyperparameter values for the machine learning classifier;
selecting a first combination of hyperparameters values from the matrix of candidate hyperparameter values and configuring the machine learning classifier using the selected first combination of hyperparameter values;
dividing the training data into N subsets, where N is an integer greater than 1;
training the machine learning classifier using N−1 subsets of the N subsets and validating the trained machine learning classifier using a remaining subset of the N subsets as a validation subset;
repeating the training and validation of the machine learning classifier for multiple rounds, wherein each round employs a different subset of the N subsets as the validation subset;
determining the optimal model parameters for the configured machine learning classifier based on the repeated training and validation;
selecting a second combination of hyperparameters from the matrix of candidate hyperparameter values and reconfiguring the machine learning classifier using the second combination of hyperparameters;
performing the multiple iterations of training on the reconfigured machine learning classifier;
repeating the selection of additional combinations of hyperparameters for training and validating until all combinations of hyperparameters are selected; and
determining an optimal mode of the machine learning classifier with the optimal hyperparameters and the optimal model parameters that generate a best validation result.

14. A method of deploying the optimal ARDS classifier determined according to the method of claim 1 for ARDS sub-phenotyping and personalized ARDS treatment.

15. The method of claim 14, wherein the deploying the optimal ARDS classifier for ARDS sub-phenotyping and providing personalized treatment comprises:
hosting the optimal ARDS classifier on a cloud platform and exposing a plurality of application programming interfaces (APIs) of the optimal ARDS classifier, wherein the plurality of APIs comprises a set of input APIs for users to input ARDS patient data and a set of output APIs for outputting predicted ARDS sub-phenotype classifications.

16. The method of claim 14, wherein the deploying the optimal ARDS classifier for ARDS sub-phenotyping and providing personalized treatment comprises:
incorporating the optimal ARDS classifier into an Electronic Medical Record (EMR) system comprising a plurality of ARDS patient medical records; and
displaying a user interface (UI) element for entering a plurality of clinical variables of the ARDS patient medical records into the optimal ARDS classifier and receiving an ARDS sub-phenotype classification result.

17. The method of claim 14, wherein deploying comprises associating an ARDS patient with an ARDS sub-phenotype.

18. The method of claim 14, wherein the ARDS sub-phenotype is a Class 1 sub-phenotype associated with a high mortality risk or a Class 2 sub-phenotype associated with a low mortality risk.

19. The method of claim 14, wherein the deploying the optimal ARDS classifier for ARDS sub-phenotyping and providing personalized ARDS treatment comprises:
storing, in a database: (1) an ARDS sub-phenotype classification result generated by the optimal classifier based on the values of a plurality of clinical variables of the ARDS patient, and (2) explanatory information of the ARDS sub-phenotype classification result; and
displaying a dashboard that visualizes (i) profile information of the ARDS patient, (ii) an ARDS sub-phenotype classification result for the ARDS patient, and (iii) a graphic user interface (GUI) comprising: (a) a first user interface (UI) element for providing access to the stored explanatory information, and (b) one or more additional UI elements for providing additional information on the ARDS sub-phenotype classification result.

20. The method of claim 14, wherein before deploying the optimal ARDS classifier, the method further comprises:
obtaining clinical data and physiological data from a plurality of randomized controlled ARDS clinical trial patient cohorts;
inputting the physiological data into the optimal ARDS classifier to obtain a predicted classification result; and
verifying the predicted classification result based on the clinical data to validate the optimal ARDS classifier.

21. The method of claim 20, wherein,
the clinical data comprises age, gender, SAPS II score, SFA score, corticosteroids, Vaso_use_24h, vasopressor use, corticosteroid use, or a combination of any of the foregoing; and
the physiological data comprises heart rate, temperature, mean arterial blood pressure, SPO₂, blood urea nitrogen, albumin, bilirubin, serum creatinine, whole blood creatinine, serum glucose, whole blood glucose, serum hematocrit, whole blood hematocrit, PCO₂ platelets, sodium, arterial TCO₂, venous TCO₂, white blood cell count, or a combination of any of the foregoing.

22. The method of claim 14, wherein the deploying the optimal ARDS classifier for ARDS sub-phenotyping comprises in response to input point-of-care variable values of an ARDS patient, generating a plurality of confidence scores respectively corresponding to a number of ARDS sub-phenotypes.

23. The method of claim 1, further comprising:
temporally tracking the values of the point-of-care variables of an ARDS patient and a series of ARDS sub-phenotype classification results generated by the optimal ARDS classifier for the ARDS patient; and
generating an alert or notification when the tracked values of the point-of-care variables of the ARDS patient cause the optimal ARDS classifier to generate a new ARDS sub-phenotype classification result that is different from an immediately preceding ARDS sub-phenotype classification result.

24. The method of claim 23, wherein the notification comprises a recommended ARDS treatment.

25. The method of claim 23, further comprising:
temporally tracking gradients of the values of the point-of-care variables of the ARDS patient, wherein the ARDS patient is undergoing an ARDS treatment regimen;
predicting future values of the point-of-care variables of the ARDS patient based on the tracked gradients;
inputting the predicted future values of the point-of-care variables of the ARDS patient into the optimal ARDS classifier to predict a future ARDS sub-phenotype classification result for the ARDS patient; and
generating an alert or notification when the predicted future ARDS sub-phenotype classification result is different than a current ARDS sub-phenotype classification result.

26. The method of claim 1, the method further comprising:
obtaining training data, wherein the training data comprises values for a plurality of clinical variables of the ARDS patient and additional information; and
retraining the optimal ARDS classifier using the obtained training data and the additional information.

27. The method of claim 26, wherein the obtaining training data comprises:
constructing and executing a first Structured Query Language (SQL) query on a first database comprising ARDS patient records to identify ARDS patients with a first set of parameters for diagnosing ARDS;
for the identified ARDS patients, constructing and executing a second SQL query on a second database comprising free-text report notes associated with ARDS patient candidate radiography imaging reports;
executing a Natural Language Processing (NLP) model against the free-text report notes to identify ARDS patients with a second set of parameters that meet criteria consistent with a clinically recognized definition of ARDS, wherein the NLP model is trained to identify keywords with synonyms using supervised learning; and
obtaining the ARDS patient monitoring records for the identified ARDS patients as the training data.

28. The method of claim 1, wherein the clinically recognized definition of ARDS comprises the ARDS New Global Definition 2023.

29. An optimal ARDS classifier determined according to the method of claim 1.

30. An ARDS sub-phenotype classification of an ARDS patient determined using the optimal ARDS classifier of claim 29.
